(19) United States
(12) Patent Application Publication
(10) Pub. No.: US 2019/0161808 A1
(43) Pub. Date: May 30, 2019

(54) METHOD FOR PREDICTING PROGNOSIS OF BREAST CANCER PATIENTS BY USING GENE DELETIONS

(71) Applicant: NGENEBIO, Seoul (KR)

(72) Inventors: Young Kee SHIN, Seoul (KR); Hae Min JEONG, Seoul (KR); Ryongnam KIM, Seoul (KR)

(21) Appl. No.: 16/300,927

(22) PCT Filed: May 12, 2017
(86) PCT No.: PCT/KR2017/004959

§ 371 Date: Nov. 12, 2018

(30) Foreign Application Priority Data
May 12, 2016 (KR) 10-2016-0058314

(51) Int. Cl. C12Q 1/6886; C12Q 1/6806; C12Q 1/686; C12Q 1/02

(52) U.S. Cl. CPC C12Q 1/6886; C12Q 1/6806; G01N 2800/52; C12Q 1/025; C12Q 1/686

(57) ABSTRACT

The present invention relates to a method for predicting the prognosis of breast cancer patients by using gene deletions and, more particularly, to: a method for detecting a marker for the prognosis of triple negative breast cancer patients in order to provide information necessary for the breast cancer prognosis diagnosis, comprising the steps of obtaining a sample of a subject, extracting genomic DNA from the sample, examining deletions of genes in the extracted genomic DNA, and determining that a subject, in which gene deletions in genomic DNA are confirmed, has a poor prognosis for breast cancer; and a composition for predicting the prognosis of breast cancer patients, containing a preparation enabling the examination of gene deletions and a kit comprising the same as an active ingredient. As investigated by the present inventors, deletions of a plurality of specific genes in triple negative breast cancer tissues are closely correlated with the prognosis of breast cancer patients, and thus the method and composition, of the present invention, which are for detecting deletions of relevant genes as a marker, are useful in providing information for determining the prognosis of breast cancer, particularly triple negative breast cancer for which efficient biomarkers are absent.

### TECHNICAL FIELD

The present application claims priority from Korean Patent Application No. 10-2016-0058314, filed on May 12, 2016, the entire content of which is incorporated herein by reference.

The present invention relates to a method for predicting the prognosis of a breast cancer patient using the deletion of a gene, more specifically, for the purpose of providing information necessary for diagnosing the prognosis of breast cancer, a method for detecting a marker of a prognosis of a breast cancer patient, the method comprising obtaining a sample of a test subject; extracting genomic DNA from the sample; confirming the presence or absence of the deletion of a gene in the extracted genomic DNA; and determining that the test subject has a breast cancer with a poor prognosis in case the presence of the deletion of a gene is confirmed in the genomic DNA; a composition for predicting the prognosis of a breast cancer patient comprising an agent capable of confirming the deletion of a gene; and a kit containing the composition as an effective ingredient.

### BACKGROUND OF THE INVENTION

Breast cancer is one of the most prevalent cancers worldwide, with over 1,300,000 newly diagnosed patients and 450,000 deaths each year. Breast cancer is a highly heterogeneous disease with diverse pathophysiological and clinical features that can be caused by distinct genetic, epigenetic, or transcriptomic changes. According to gene and protein expression profiles, breast cancer can be classified as luminal A type, luminal B type, HER2+ type and triple negative breast cancer (TNBC), respectively. TNBC is defined as a tumor that is deficient in the expression of estrogen receptor (ER), progesterone receptor (PR), and human epidermal growth factor receptor 2 (HER2). TNBC accounts for approximately 10-20% of invasive breast cancers, and the mortality rate of women with TNBC increases over the 5 years after diagnosis.

Luminal A, B and HER2 types of breast cancer can be treated with hormone therapy and HER2 receptor target therapy, respectively, but no therapeutic effects of these therapies are expected for TNBC because there is no receptor (ER, PR, HER2) that is the target of these therapies. There have been several pioneering genome-wide studies that are aimed to identify diagnostic and therapeutic biomarkers in TNBC, but there has been no comprehensive effort to date that has attempted to develop TNBC biomarkers for Koreans to date.

In recent years, a targeted exome next generation sequencing (NGS) analysis technique for analyzing a target exome region of cancer genome, which is excellent in terms of cost-effectiveness compared with significantly facilitated whole genome or next-generation sequencing (NGS) of whole genome or whole exome, has human clinical cancer diagnosis, studies on cancer-causing mechanisms, and the identification of therapeutic targets. Since the targeted exome NGS can provide an in-depth readings on the sequence of the targeted exome region at a relatively low cost as compared to the whole exome NGS, it is very advantageous in carrying out analysis on mutation and copy number variation in a more reliable manner. In particular, it is already known that the HaloPlex target enrichment system is very effective in capturing targeted regions on the exome, thus being very useful for the targeted exome NGS.

Therefore, it is necessary to utilize the above technology to find a biomarker for diagnosis and treatment of breast cancer, especially TNBC, suitable for Korean population.

### DETAILED DESCRIPTION OF THE INVENTION

### Technical Problem

Accordingly, the present inventors have performed exome sequencing of target genes associated with cancer in order to develop a gene marker capable of diagnosing the prognosis of breast cancer patients, particularly triple negative breast cancer patients, comprising the present invention by confirming that the deletion of multiple genes are closely related to the survival rate of breast cancer patients.

Accordingly, an aspect of the present invention is to provide a method for detecting a marker of a prognosis of a breast cancer patient, the method comprising;

obtaining a sample of a test subject;

extracting genomic DNA from the sample;

confirming the presence or absence of the deletion of a gene in the extracted genomic DNA; and

determining that the test subject has a breast cancer with a poor prognosis in case the presence of the deletion of a gene is confirmed in the genomic DNA.

Another aspect of the present invention is to provide a composition for predicting the prognosis of a breast cancer patient, the composition comprising an agent capable of confirming the deletion of a gene.

Also, another aspect of the present invention is to provide a composition for predicting the prognosis of a breast cancer patient, the composition consisting of an agent capable of confirming the deletion of a gene.

Also, another aspect of the present invention is to provide a composition for predicting the prognosis of a breast cancer patient, the composition consisting essentially of an agent capable of confirming the deletion of a gene.

Another aspect of the present invention is to provide a kit comprising the composition for predicting the prognosis of a breast cancer patient, the composition comprising an agent capable of confirming the deletion of a gene as an active ingredient.

Another aspect of the present invention is to provide use of an agent capable of confirming the deletion of a gene for preparing an agent for predicting the prognosis of a breast cancer patient.

### Technical Solution

An embodiment according to an aspect of the present invention provides a method for detecting a marker of a prognosis of a breast cancer patient, the method comprising;

obtaining a sample of a test subject;

extracting genomic DNA from the sample;

confirming the presence or absence of the deletion of a gene in the extracted genomic DNA; and

determining that the test subject has a breast cancer with a poor prognosis in case the presence of the deletion of a gene is confirmed in the genomic DNA.

An embodiment according to an aspect of the present invention provides a composition for predicting the prognosis of a breast cancer patient, the composition comprising an agent capable of confirming the deletion of a gene.

Also, an embodiment according to another aspect of the present invention provides a composition for predicting the prognosis of a breast cancer patient, the composition consisting of an agent capable of confirming the deletion of a gene.

Also, an embodiment according to another aspect of the present invention provides a composition for predicting the prognosis of a breast cancer patient, the composition consisting essentially of an agent capable of confirming the deletion of a gene.

An embodiment according to an aspect of the present invention provides a kit comprising the composition for predicting the prognosis of a breast cancer patient, the composition comprising an agent capable of confirming the deletion of a gene as an active ingredient.

An embodiment according to an aspect of the present invention provides use of an agent capable of confirming the deletion of a gene for preparing an agent for predicting the prognosis of a breast cancer patient.

Hereinafter, the present invention will be described in detail.

The present invention provides a method for detecting a marker of a prognosis of a breast cancer patient, the method comprising;

obtaining a sample of a test subject;

extracting genomic DNA from the sample;

confirming the presence or absence of the deletion of a gene in the extracted genomic DNA; and

determining that the test subject has a breast cancer with a poor prognosis in case the presence of the deletion of a gene is confirmed in the genomic DNA.

The method for detecting the marker of prognosis of a breast cancer patient according to the method of the present invention is aimed to provide information necessary for diagnosing the prognosis of breast cancer, and is most preferably applied to a triple-negative breast cancer (TNBC) patient.

As used herein, the term ‘triple-negative breast cancer (TNBC)’ refers to a breast cancer in which estrogen receptors (ER), progesterone receptors (PR) and human epidermal growth factor receptor2 (HER2), which are hormone receptors, are not expressed in breast cancer tissue, among four molecular types of breast cancer classified according to the expression of hormone receptor and HER2. TNBC is sometimes classified as a ‘basal-type’ while there are no established classification criteria. Basal-type cancer is defined as cytokeratin 5/6 and epidermal growth factor receptor (EGFR) staining, which is not yet an established criteria. It is estimated that about 75% of basal-type breast cancers are TNBC (Hudis C A et al., Oncologist, Suppl 1:1-11, 2011).

In order to determine the prognosis of breast cancer according to the method of the present invention, at least one gene selected from the group consisting of ATM, CHUK, EPHA5, LIFR, EBF1, NR4A3, MITF, TRIM33, MAP2K4, BMPR1A, CDK8, MDM2, EXT1, ACSL3, STK36, HMGA2, RUNX1T1, TLR4, ERCC5, THOC5, IDH2 [symbol] HNRNPA2B1 are analyzed for the presence absence of the deletion of said gene. One of these genes may be selected, and two or more genes in combination may be selected to predict breast cancer prognosis based on the presence or absence of the deletion of said gene.

In the present invention, the ‘ATM’ gene is an abbreviation of Ataxia telangiectasia mutated, which encodes serine/threonine kinase activated by DNA double strand break (DSB), and also is referred to as AT1, ATA, ATC, ATD, ATE, ATDC, TEL1, TELO1 and the likde. When DSB damage occurs in DNA, it phosphorylates key proteins involved in DNA damage, such as p53, CHK2, and BRCA1, thereby stopping the cell cycle and playing a role in inducing DNA repair or apoptosis. In humans, the ATM gene is located on chromosome 11 (11q22-q23; 108.22 to 108.37 Mb), and the nucleotide sequence of the genomic DNA in which the ATM gene is located can be found in Genbank accession no. NC_000011.10 (108222500˜108369102 bp), the mRNA of the ATM gene is Genbank accession no. NM_000051.3 (13147 bp), and the like. The ATM gene is known to consist of about 63 exons.

In the present invention, the ‘CHUK’ gene encodes a protein kinase called inhibitor of nuclear factor kappa-B kinase subunit alpha (IKK-α), conserved helix-loop-helix ubiquitous kinase, IKK1, IKKA, IKBKA, TCF16, NFKBIKA, IKK-alpha and the like. In humans, it is located at 10q24-q25 on chromosome 10 and consists of about 23 exons. The nucleotide sequence of the genomic DNA in which the CHUK gene is located is known as NC_000010.11 (100186113-100229610 bp), and the mRNA is known as Genbank accession number such as NM_001278.4 (3628 bp).

In the present invention, the ‘EPHA5’ gene encodes a protein belonging to the ephrin receptor subfamily known as EPH receptor A5, ephrin type-A receptor 5, EK7, CEK7, EHK1, HEK7, EHK-1, TYRO4 and the like. In humans, it is located at chromosome 4q13.1 on chromosome 4 and consists of about 21 exons. The nucleotide sequence of the genomic DNA in which the EPHA5 gene is located is known as NC_000004.12 (65319563˜65670495 bp), and the nucleotide sequence of the mRNA is known as Genbank accession number such as NM_001281765.2 (8438 bp).

In the present invention, the ‘LIFR’ gene encodes a subunit of the LIF receptor known as a leukemia inhibitory factor receptor, a leukemia inhibitory factor receptor alpha, SWS, SJS2, STWS, CD118, LIF-R and the like. In humans, it is located at 5p13-p12 on chromosome 5 and consists of about 24 exons. The nucleotide sequence of the genomic DNA in which the LIFR gene is located is known as NC_000005.10 (38474963 to 38595405 bp), and the mRNA nucleotide sequence is known as Genbank accession number such as NM_001127671.1 (10258 bp).

In the present invention, the ‘EBF1’ gene encodes a protein known as transcription factor COE1 or early B-cell factor 1, COE1, EBF, O/E-1, OLF1 and the like. In humans, it is located at 5q33.3 on chromosome 5 and consists of about 22 exons. The nucleotide sequence of the genomic DNA in which the EBF1 gene is located is known as Genbank accession no. NC_000008.11 (31033262˜31173761 bp), and the mRNA of the EBF1 gene is known as NM_001290360.2 (5267 bp).

In the present invention, the ‘NR4A3’ gene encodes a protein known as neuron-derived orphan receptor 1 (NOR1), CHN, CSMF, MINOR, TEC and the like. In humans, it is located at 9q31.1 on chromosome 9 and consists of about 10 exons. The nucleotide sequence of the genomic DNA in which the NR4A3 gene is located is known as NC_000009.12 (99821855˜99866893 bp), and the mRNA of the NR4A3 gene is known as Genbank accession no. NM_006981.3 (5635 bp).

In the present invention, the ‘MITF’ gene encodes a protein known as class E basic helix-loop-helix protein 32, a microphthalmia-associated transcription factor, bHLHe32, CMM8, COMMAD, MI, WS2, WS2A and the like. In humans, it is located at 3p13 on chromosome 3 and consists of about 17 exons. The nucleotide sequence of the genomic DNA in which the MITF gene is located is known as NC_000003.12 (69739435 . . . 69968337 bp), and the mRNA of the MITF gene is known as Genbank accession number such as NM_000248.3 (4472 bp).

In the present invention, the ‘TRIM33’ gene encodes a protein known as Tripartite motif-containing 33 (TRIM33) which is known as transcriptional intermediary factor 1 gamma (TIF1-), ECTO, PTC7, RFG7, TF1G, TIF1G, TIF1GAMMA, TIFGAMMA and the like. In humans, it is located at 1p13.2 on chromosome 1 and consists of about 21 exons. The nucleotide sequence of the genomic DNA in which the TRIM33 gene is located is known as NC_000001.11 (114392777˜114511160 bp), and the mRNA of the TRIM33 gene is known as Genbank accession number such as NM_015906.3 (8339 bp).

The ‘MAP2K4’ gene in the present invention encodes a transcription factor called Dual specificity mitogen-activated protein kinase kinase 4, and JNKK, JNKK1, MAPKK4, MEK4, MKK4, PRKMK4, SAPKK-1, SAPKK1, SEK1, SERK1, SKK1 and the like. In humans, it is located at 18q12 on chromosome 17 and consists of about 15 exons. The nucleotide sequence of the genomic DNA in which the MAP2K4 gene is located is known as NC_000017.11 (12020818˜12143831 bp), and the mRNA of the MAP2K4 gene is known as Genbank accession number such as NM_001281435.1 (3873 bp).

In the present invention, the ‘BMPR1A’ gene encodes a protein known as bone morphogenetic protein receptor, type IA, ACVRLK3, ALK3, CD292, SKR5, and the like. In humans, it is located at 10q23.2 on chromosome 10 and consists of about 15 exons. The nucleotide sequence of the genomic DNA in which the BMPR1A gene is located is known as NC_000010.11 (86755786˜86927969 bp), and the mRNA of the BMPR1A gene is known as Genbank accession number such as XM_011540103.2 6294 bp).

In the present invention, the ‘CDK8’ gene encodes a protein known as Cell division protein kinase 8 and K35. In humans, it is located at 13q12.13 on chromosome 13 and consists of about 15 exons. The nucleotide sequence of the genomic DNA in which the CDK8 gene is located is known as NC_000013.11 (26254104˜26405238 bp), and the mRNA of the CDK8 gene is known as Genbank accession number such as NM_001260.2 (3101 bp).

In the present invention, ‘MDM2’ gene encodes a mouse double minute 2 homologue known as E3 ubiquitin-protein ligase Mdm2, ACTFS, HDMX, hdm2 and the like. In humans, it is located at 12q15 on chromosome 12 and consists of about 13 exons. The nucleotide sequence of the genomic DNA in which the MDM2 gene is located is known as NC_000012.12 (68808149˜68845544 bp), and the mRNA of the MDM2 gene is known as Genbank accession number such as NM_001145337.2 (7104 bp).

In the present invention, the ‘PLCG2’ gene encodes a phospholipase protein known as 1-phosphatidylinositol-4,5-bisphosphate phosphodiesterase gamma-2, phospholipase C gamma 2, FCAS3, APLAID, PLC-IV, PLC-gamma-2 and the like. In humans, it is located at 16q24.1 on chromosome 16 and consists of about 25 exons. The nucleotide sequence of the genomic DNA in which the PLCG2 gene is located is known as NC_000016.10 (81779258˜81962693 bp), and the mRNA of the PLCG2 gene is known as Genbank accession number such as NM_002661.4 (8707 bp).

In the present invention, the ‘EXT1’ gene encodes a protein known as Exostosin-1, MEXT, LGCR, LGS, TRPS2, TTV and the like. In humans, it is located at 8q24.11 on chromosome 8 and consists of about 12 exons. The nucleotide sequence of the genomic DNA in which the EXT1 gene is located is known as NC_000008.11 (117797496˜118111819 bp), and the mRNA of the EXT1 gene is known as Genbank accession number such as XR_001745492.1 (3790 bp).

In the present invention, the ‘ACSL3’ gene encodes a protein known as long-chain-fatty-acidCoA ligase 3, ACS3, FACL3, PRO2194 and the like. In humans, it is located at 2q36.1 on chromosome 2 and consists of about 17 exons. The nucleotide sequence of the genomic DNA in which the ACSL3 gene is located is known as NC_000012.12 (49018975˜49061895 bp), and the mRNA of the ACSL3 gene is known as Genbank accession number such as NM_004457.3 (4369 bp).

In the present invention, the ‘STK36’ gene encodes an enzymatic protein which is serine/threonine-protein kinase 36. In humans, it is located at 2q35 on chromosome 2 and consists of about 30 exons. The nucleotide sequence of the genomic DNA in which the STK36 gene is located is known as NC_000002.12 (218672026˜218702717 bp), and the mRNA of the STK36 gene is known as Genbank accession number such as NM_001243313.1 (4883 bp).

In the present invention, ‘HMGA2’ gene encodes a protein known as high-mobility group AT-hook 2, BABL, HMGI-C, HMGIC, LIPO, STQTL9 and the like. In humans, it is located at 12q14.3 on chromosome 12 and consists of about 8 exons. The nucleotide sequence of the genomic DNA in which the HMGA2 gene is located is known as NC_000012.12 (65824460˜65966291 bp), and the mRNA of the HMGA2 gene is known as Genbank accession number such as NM_001300918.1 (1274 bp).

In the present invention, the ‘RUNX1T1’ gene encodes a protein known as Protein CBFA2T1, AML1-MTG8, AML1T1, CBFA2T1, CDR, ETO, MTG8, ZMYND2 and the like. In humans, it is located at 8q21.3 on chromosome 8 and consists of about 20 exons. The nucleotide sequence of the genomic DNA in which the RUNX1T1 gene is located is known as NC_000008.11 (91954967˜92103365 bp), and the mRNA of the RUNX1T1 gene is known as Genbank accession number such as NM_001198625.1 (7769 bp).

In the present invention, the ‘TLR4’ gene encodes a protein known as Toll-like receptor 4, ARMD10, CD284, TLR-4, TOLL and the like. In humans, it is located at 9q33.1 on chromosome 9 and consists of about 4 exons. The nucleotide sequence of the genomic DNA in which the TLR4 gene is located is known as NC_000009.12 (117704175˜117717491 bp), and the mRNA of the TLR4 gene is known as Genbank accession number such as NM_003266.3 (5781 bp).

In the present invention, the ‘ERCC5’ gene encodes a protein known as ribosomal protein S6 kinase alpha-2, ribosomal protein S6 kinase A2, COFS3-201, ERCM2, UVDR, XPG, XPGC, ERCC5 and the like. In humans, it is located at 13q33.1 on chromosome 13 and consists of about 15 exons. The nucleotide sequence of the genomic DNA in which the ERCC5 gene is located is known as NC_000013.11 (102845841 . . . 102876001 bp), and the mRNA of the ERCC5 gene is known as Genbank accession number such as NM_000123.3 (4091 bp).

In the present invention, the ‘THOC5’ gene encodes a protein known as rTHO complex subunit 5 homolog, C22orf19, Fmip, PK1.3, fSAP79 and the like. In humans, it is located at 22q12.2 on chromosome 22 and consists of about 23 exons. The nucleotide sequence of the genomic DNA in which the THOC5 gene is located is known as NC_000022.11 (29508167˜29554254 bp), and the mRNA of the THOC5 gene is known as Genbank accession number such as NM_001002877.1 (2563 bp).

In the present invention, ‘IDH2’ gene encodes a protein known as rIsocitrate dehydrogenase [NADP], mitochondrial, D2HGA2, ICD-M, IDH, IDHM, IDP, IDPM, mNADP-IDH and the like. In humans, it is located at 15q26.1 on chromosome 15 and consists of about 12 exons. The nucleotide sequence of the genomic DNA in which the IDH2 gene is located is known as NC_000015.10 (90083978˜90102554 bp), and the mRNA of the IDH2 gene is known as Genbank accession number such as NM_001289910.1 (1578 bp).

In the present invention, ‘HNRNPA2B1’ gene encodes a protein known as Heterogeneous nuclear ribonucleoproteins A2/B1, HNRNPA2, HNRNPB1, HNRPA2, HNRPA2B1, HNRPB1, IBMPFD2, RNPA2, SNRPB1 and the like. In humans, it is located at 7p15.2 on chromosome 7 and consists of about 13 exons. The nucleotide sequence of the genomic DNA in which the HNRNPA2B1 gene is located is known as NC_000007.14 (26189927˜26200793 bp), and the mRNA of the HNRNPA2B1 gene is known as Genbank accession number such as NM_002137.3 (3666 bp).

According to the present inventors, the deletion of the above described genes is closely related to the prognosis of breast cancer, particularly TNBC breast cancer. In one embodiment of the present invention, targeted exome sequencing was performed on genes selected using a sample obtained from a patient with TNBC in order to identify genetic markers useful for the prognosis prediction and treatment of breast cancer patients. Exome sequencing was performed on genomic DNA extracted from the samples of breast cancer tissues and normal tissues from 70 Korean TBBC patients. As a result, the deletion of a gene in ATM, CHUK, EPHA5, LIFR, EBF1, NR4A3, MITF, TRIM33, MAP2K4, BMPR1A, CDK8, MDM2, EXT1, ACSL3, STK36, HMGA2, RUNX1T1, TLR4, ERCC5, THOC5, IDH2 and HNRNPA2B1 genes was found in breast cancer tissues.

According to another embodiment of the present invention, the deletion of the gene and the survival rate of TNBC breast cancer patients are closely related. In case of TNBC patients with homozygous deletion in the gene, there was found a higher probability of recurrence and distant metastasis, with significantly less disease free survival (DFS), in comparison with patients without homozygous deletion. Also, Kaplan-Meier survival curve analysis showed that patients with homozygous deletion of the genes had a short survival period, confirming that the homozygous deletion of the genes and the prognosis of TNBC were inversely correlated.

Thus, one of ordinary skill in the art can understand that the correlation between the deletion of a gene identified by the present inventors and the TNBC prognosis can be used to provide information necessary for detecting the prognosis of breast cancer, particularly TNBC prognosis.

As used herein, the term ‘prognosis’ refers to a prospect of a future symptom or progress which is judged by diagnosis of a disease. For cancer patients, the prognosis usually refers to the recurrence of a cancer, or the metastasis of a cancer or survival period within a period certain of time after surgical procedure. Prediction of prognosis (or diagnosis of prognosis) is a very important clinical task, especially because it provides clues to the future direction of breast cancer treatment, including the chemotherapy of early breast cancer patients. The prediction of prognosis also includes the prediction of the patient's response to therapies and the progression of therapies.

Herein, as a marker for determining the prognosis of breast cancer, more specifically, TNBC, the deletion of a gene is preferably the deletion of an exon which is a part of a gene encoding a protein. The deletion of a gene may be the deletion of one or more exons that constitutes the gene, while the extent of the deletion in length is not limited. One or more exons may be all deleted. For example, the deletion of the ATM gene may occur in one or more of 63 exons. Also, for more accurate prognosis judgment, it is preferable that the deletion of a gene is the homozygous deletion of the ATM gene in which alleles of the gene are all deleted.

Specifically, the sample for determining the presence or absence of the deletion of a gene is obtained from breast cancer tissues. In order to confirm the mutation of genomic DNA in breast cancer tissues, non-cancerous, normal tissues, around breast cancer tissues or areas corresponding to breast cancer tissues may be additionally collected from the same test subject. Unless limiting the extraction of the genome DNA from the subject and the analysis of the deletion of a gene, the sample may be pre-treated for storage or other analysis, for example, immunohistochemical staining. For genomic DNA analysis, the sample is preferably a fresh sample or a rapidly frozen sample, but may be a formalin-fixed paraffin-embedded (FFPE) tissue.

TNBC may be farther confirmed among breast cancers by performing a step of confirming the absence of the expression of the estrogen receptor, progesterone receptor and HER2 gene in a sample of breast cancer tissue collected from the breast cancer patient. At this time, the absence of the gene expression may be confirmed by the absence of the mRNA or protein of the gene by a known method.

The presence or absence of the deletion of the gene may be carried out by any conventional method without any limitations to detect a small insertion or deletion (INDEL) of a specific gene in a genomic DNA (gDNA). Since a copy number variation (CNV) may be induced when the deletion site of a gene is large, it is also possible to confirm the presence or absence of the deletion of a gene using a method of detecting the copy number variation. Specifically, a method of detecting the marker according to the present invention can be carried out by appropriately selecting a method among sequencing-based methods such as direct sequencing, next generation sequencing, targeted exome sequencing, sequencing read depth method, whole genome sequence assembly; polymerase chain reaction (PCR)-based methods such as quantitative PCR), multiplex amplifiable probe hybridization (MAPH), multiplex ligation-dependent probe amplification (MLPA), paralogue ratio test (PRT); DNA array-based methods such as array comparative genomic hybridization (array CGH), SNP microarray; hybridization-based methods such as fiber FISH, southern blotting and pulsed field gel electrophoresis (PFGE), or the like. More detailed descriptions of these methods can be found in the literature (Cantsilieris S et al., Genomics, 101(2):86-93, 2013).

A person skilled in the art, in carrying out the above method, the suitable position and nucleotide sequence of a primer or a probe necessary for confirming the deletion of a specific gene can be selected according to a known method using known nucleotide sequence information of the gene and gDNA around the gene.

Also, the present invention provides a composition for predicting the prognosis of a breast cancer patient, the composition comprising an agent capable of confirming the deletion of a gene.

Also, the present invention provides a composition for predicting the prognosis of a breast cancer patient, the composition consisting of an agent capable of confirming the deletion of a gene.

Also, the present invention provides a composition for predicting the prognosis of a breast cancer patient, the composition consisting essentially of an agent capable of confirming the deletion of a gene.

The composition for predicting the prognosis of the breast cancer patient is most preferably applied to determine the prognosis of a triple negative breast cancer (TNBC) patient.

Specifically, the gene for confirming the deletion of a gene by the composition for predicting the prognosis of a breast cancer patient according to the present invention may be at least one gene selected from the group consisting of ATM, CHUK, EPHA5, LIFR, EBF1, NR4A3, MITF, TRIM33, MAP2K4, BMPR1A, CDK8, MDM2, EXT1, ACSL3, STK36, HMGA2, RUNX1T1, TLR4, ERCC5, THOC5, IDH2 and HNRNPA2B1. The composition according to the present invention may confirm the deletion of a single gene or two or more genes in combination among the above described genes.

The composition specifically comprises an agent necessary for carrying out a method for confirming the deletion of a specific gene. Methods for determining the deletion of a gene may be based on a variety of techniques such as sequencing, PCR, hybridization, and arrays, as described above. The agent capable of confirming deletion of a specific gene may particularly be a specific primer pair or a probe of the gene. The primer or the probe may be labeled with fluorescence, radioactive isotope or the like

In addition, the present invention provides a kit comprising the composition for predicting the prognosis of a breast cancer patient as an active ingredient, the composition comprising an agent capable of confirming the deletion of a gene.

The kit according to the present invention comprise, as an active ingredient, a composition for predicting the prognosis of a breast cancer patient which comprises an agent capable of confirming the deletion of the gene described above. It farther includes other components necessary to confirm the deletion of the gene, such as buffers, coenzymes, enzyme substrates, positive control DNA, etc. necessary to carry out experimental methods for identifying the deletion of the gene. The kit is a constituent unit for detecting the deletion of a gene from the genomic DNA extracted from a sample of a subject as a marker of a breast cancer prognosis.

Also, the present invention provides use of an agent capable of confirming the deletion of a gene for preparing an agent for predicting the prognosis of a breast cancer patient.

As used herein, the term ‘an agent capable of confirming the deletion of a gene’ is the same as described above, while the gene for confirming the deletion of a gene is the same as described above, i.e, the gene is at least one gene selected from the group consisting of ATM, CHUK, EPHA5, LIFR, EBF1, NR4A3, MITF, TRIM33, MAP2K4, BMPR1A, CDK8, MDM2, EXT1, ACSL3, STK36, HMGA2, RUNX1T1, TLR4, ERCC5, THOC5, IDH2 and HNRNPA2B1.

In one embodiment of the present invention, the prognosis of breast cancer patient (a breast cancer patient of TNBC) who have undergone chemotherapy, particularly adjuvant chemotherapy, depends on the deletion of a gene according to the present invention, leading to different responsiveness to chemotherapy.

Therefore, the present invention provides a method for predicting the responsiveness of a breast cancer patient to chemotherapy, the method comprising:

(a) obtaining a sample of a test subject undergoing chemotherapy;

(b) extracting genomic DNA from the sample;

(c) confirming the presence of absence of the deletion of a gene in the extracted genomic DNA; and

(d) determining that the test subject has a breast cancer with a poor prognosis in case the presence of the deletion of a gene is confirmed in the genomic DNA.

As used herein, the term chemotherapy (or chemical therapy) in the present invention refers to a use of a chemotherapeutic reagent for the treatment of cancer, tumor or malignant neoplasm formation, the term ‘chemotherapeutic agent’ refers to a compound used in chemotherapy, particularly those which damage mitosis (cell division) by effectively targeting rapidly dividing cells. Some chemotherapeutic agents induce apoptosis (so-called “cell suicide”) in cells. Preferred chemotherapeutic agents herein may platin-derived agents, plant alkaloids and terpene, and more preferably, may include Vincristin, vinblastin, Vinorelbine, Vindesine, Paclitaxel, Docetaxel, Anastrozole, Bicalutamide, Buserelin, Capecetabine, Cisplatin, Carboplatin, Desoxorubicin, Etoposide, Fulvestrant, Gemcitabine, Goserelin, Irionotecan, Letrozole, Leuproreline, Megestrol, Mitotoane, Mitoxantrone, Oxalipatin, Pemetrexed, Raltitrexed, Tamoxifen, Tegafur and Triptoreline.

The chemotherapy of the present invention may be an adjuvant chemotherapy, which means an additional cancer treatment after the first treatment to lower the risk of cancer reoccurrence.

The prediction of the responsiveness to chemotherapy as described above may be performed by detecting the deletion of the gene in the genomic DNA. Therefore, the method of predicting the responsiveness of the breast cancer patient to chemotherapy according to the present invention may be a method of detecting the deletion of the gene or a method of detecting the deletion of the gene in the genomic DNA. In this case, the method may comprise the steps (a) to (c).

In addition, the present invention provides a composition for predicting the responsiveness of a breast cancer patient to chemotherapy, the composition comprising an agent capable of confirming the deletion of a gene, and further, provides use of an agent capable of confirming the deletion of a gene for preparing an agent for predicting the responsiveness of a breast cancer patient to chemotherapy.

The term “comprising” is used synonymously with “containing” or “being characterized”, and does not exclude additional ingredients or steps that are not mentioned in the compositions and the methods. The term “consisting of” excludes additional elements, steps, or ingredients that are not separately described. The term “consisting essentially of” means that in the scope of the compositions or methods, the term includes any material or step that does not substantially affect basic characteristics of the compositions or methods, as well as described materials or steps.

### Advantageous Effect

Accordingly, in order to provide information necessary for diagnosing the prognosis of breast cancer, the present invention provides a method for detecting a marker of a prognosis of a breast cancer patient, the method comprising; obtaining a sample of a test subject; extracting genomic DNA from the sample: confirming the presence or absence of the deletion of a gene in the extracted genomic DNA; and determining that the test subject has a breast cancer with a poor prognosis in case the presence of the deletion of a gene is confirmed in the genomic DNA, a composition for predicting the prognosis of a breast cancer patient, the composition comprising an agent capable of confirming the deletion of a gene, and a kit comprising the same as an effective ingredient. As identified by the present inventors, since there is a close correlation between the deletion of multiple specific genes and the prognosis of breast cancer patients in triple-negative breast cancer tissues, the confirmation of the presence of absence of the deletion of a specific gene can provide information useful for determining the treatment and prognosis of breast cancer, particularly triple negative breast cancer.

### BRIEF DESCRIPTION OF DRAWINGS/FIGURES

FIG. 1 shows the clinical pathological features of 70 Korean triple negative breast cancer patients subject to targeted exome sequencing analysis.

FIGS. 2A-2D show the results of quantitative polymerase chain reaction (qPCR) of the deletions of WRN (FIG. 2A), ATM (FIG. 2B), BRCA1 (FIG. 2C), and BRCA2 (FIG. 2D) gene confirmed by exome sequencing, respectively. The serial number starting with TNBC shows the patient who have been confirmed to have a gene deletion by NGS, while N represents the normal tissue of the patient, and T represents the tumor tissue of the patient.

FIGS. 3A-3B show the result of analysis of somatic single nucleotide variants (SNV) in the genome of 70 Korean triple-negative breast cancer patients (FIG. 3A) and the result of analysis of the number of SNV and genetic copy number variation (CNV) per patient (FIG. 3B).

FIGS. 4A-4B show a summary figure of the most frequent somatic cell SNV and CNV identified in 70 Korean triple-negative breast cancer patients.

FIGS. 5A-5C shows the result of hazard ratio analysis for the deletion of the gene and prognosis identified in 70 Korean triple-negative breast cancer patients.

FIGS. 6A-6B show the results of analysis of the correlation between the homozygous deletion mutation of a gene and the prognosis of a triple-negative breast cancer patient, through DFS (FIG. 4A) and DMFS (FIG. 4B). HR, hazard ratio; CI, confidence interval.

FIGS. 7A-7B are Kaplan-Meier survival analysis diagrams showing the correlation between homozygous deletion mutants of a gene and survival probability of triple-negative breast cancer patients.

FIGS. 8A-8C are The Cancer Genome Atlas (TCGA) analysis of breast cancer data, showing the results of comparing genomic copy number and mRNA expression level of COX6C, EXT1, MYC, NBN, NDRG1 and UBR5 in clinical breast cancer samples (FIG. 8A), the results of analysis of the survival rate of breast cancer patients with gene amplification (FIG. 8B), and the results of analysis of correlation between TNBC and genes involved in DNA damage response in 70 Korean triple-negative breast cancer patients (FIG. 8C), respectively.

### MODE FOR CARRYING OUT INVENTION

Hereinafter, the present invention will be described in detail.

However, the following examples are only illustrative of the present invention, and the present invention is not limited to the following examples.

Experimental Methods

Research Ethics Statement

This study plan for analyzing cancer genomes from Korean TNBC patients was reviewed and approved by the Institutional Review Board of the Samsung Medical Center, Seoul (South Korea). All patients gave written informed consents for donating their tissues for this study. This research was performed in accordance with the principles of the Declaration of Helsinki for biomedical research with human subjects.

Target Gene Selection

Target genes included those which had been previously reported and listed as mutated in solid tumors and sarcomas in the Cancer Gene Census of the Wellcome Trust Sanger Institute (234 genes). Hematological cancer-associated genes were excluded. Genes encoding transcription factors and factors related to cell growth and kinases were selected as well (135 genes). The entire target region analyzed encompassed 961,497 bp corresponding a total of 368 genes and their 5,700 exon regions.

Next Gene Sequencing (NGS) Based on HaloPlex Target Enrichment

A total of 70 TNBC and matched normal tissues were collected at Samsung Medical Center, Seoul. Specimens were frozen immediately in liquid nitrogen or formalin-fixed and paraffin-embedded (FFPE) to produce tissue blocks for histological analysis. Genomic DNA was extracted from frozen samples using Dneasy Blood & Tissue kits (Qiagen, Hilden, Germany) according to the manufacturer's instructions. Purity of DNA was examined using the ratios of 260 nm/280 nm (between 1.8-2.1) and 260 nm/230 nm (≥1.5) following absorbance measurements by a spectrophotometer. After digestion with restriction enzymes and denaturation, target genomic DNA fragments were hybridized with biotinylated HaloPlex probes designed to guide circularization, and retrieved using magnetic streptavidin beads. Probe-bound and circularized target DNA fragments were closed by ligation and only those circularized DNA fragments were amplified by PCR, thus providing enriched and barcoded products, and subjected to sequencing analysis with Illumina HiSeq 2000.

Immunohistochemistry

FFPE tissues were sectioned and stained with hematoxylin and eosin for validation by a pathologist. Tumor tissues from TNBC patients were stained immunohistochemically for the expression of estrogen receptor (ER), progesterone receptor (PR) and human epidermal growth factor receptor 2 (HER2). Stained tissues were assessed by the pathologist and confirmed lack of the expression.

Bioinformatics Analysis of SNVs and INDELs

Paired-end sequence raw reads were trimmed and filtered to produce clean reads with good base quality (Phred Q score>20). Burrows-Wheeler Alignment (BWA 0.5.9), the Genome Analysis Toolkit (GATK), and SAMtools were used to align these paired-end sequencing reads with the human reference genome hg19. Identified SNVs and small INDELs were analyzed using the variant databases, such as dbSNP135, dbNSFP COSMIC, and the 1000 Genomes, and several software programs, such as SNPEff, SIFT, PolyPhen2, LRT, PhyloP, Mutation_Taster, Mutation_Assessor, FATHMM, and GERP_NR. Somatic non-synonymous SNVs and INDELs were selected using the following criteria: a ≥20% read-allele frequency at the position; 15 mapped reads at the position; and zero SNV or INDEL allele reads in the targeted sequence of corresponding normal tissue. Variants were confirmed by visualization in the Interactive Genomic Viewer and NextGENe software v2.3.1 (SoftGenetics, State College, Pa., USA).

Bioinformatics Analysis of CNVs

Genomic CNVs were assessed using NextGENe v2.3.1 (SoftGenetics), which compared the median read coverage levels between target genomic regions of cancer and matched normal tissues after global normalization of genome-wide read coverage levels. CNVs were calculated as the log 2 ratio of read coverage in cancer and matched normal tissues. CNVs with a log 2 ratio>1.5 were considered amplified, whereas CNVs with a log 2 ratio<−1.2 were considered homozygous loss-of-function mutations.

Experimental Validation of Genomic Alterations

Among those genes found to have deletions by NGS, WRN, ATM, BRCA1 and BRCA2 were selected for validation of CNVs by qPCR. qPCR was performed with genomic DNA from tumor and matched normal tissues of TNBC patients using primers listed in Table 1, and the results were quantified according to the ddCt method using TERT as a reference gene. DNA copy numbers of the normal tissue and tumor from the patient were compared using log 2 ratios and CNVs with a log 2 ratio 1.2 were considered a homozygous deletion.

TABLE 1
Primers used in the qPCR reactions

| name | sequence 5′-3′ |
|---|---|
| WRN_CNV_F (SEQ ID NO: 1) | CCA GGT CTC TGT GCA TTT CA |
| WRN_CNV_R (SEQ ID NO: 2) | GGT AAT ACC TGA AAA CAG GAA CTG A |
| WRN_Probe (SEQ ID NO: 3) | FAM-GAA ATG ATG AAA AAG CAA CAC A-BHQ1 |
| ATM_CNV#1_F (SEQ ID NO: 4) | GAATAATTGTTTTTATTTCTTTGTTGC |
| ATM_CNV#1_R (SEQ ID NO: 5) | TTAACAATCGCAGGAAAAAGC |
| ATM_CNV#1_probe (SEQ ID NO: 6) | FAM-TGTCTTAATTGCAGAAGAGTCCA-BHQ1 |
| ATM_CNV#2_F (SEQ ID NO: 7) | AAACAAAAGTGTTGTCTTCATGC |
| ATM_CNV#2_R (SEQ ID NO: 8) | GAACTTCTTTTTCACCAGTGTGG |
| ATM_CNV#2_probe (SEQ ID NO: 9) | FAM-TGCAGTTATCCAAGATGGCA-BHQ1 |
| BRCA1_CNV#1_F (SEQ ID NO: 10) | TTCTACAGAGTGAACCCGAAAA |
| BRCA1_CNV#1_R (SEQ ID NO: 11) | GGCTAAGGCAGGAGGACTG |
| BRCA1_CNV#1_probe (SEQ ID NO: 12) | FAM-ATGGAGTCTTGCTCTGTGGC-BHQ1 |
| BRCA1_CNV#2_F (SEQ ID NO: 13) | CAGCGATACTTTCCCAGAGC |
| BRCA1_CNV#2_R (SEQ ID NO: 4) | TTGCAAAACCCTTTCTCCAC |
| BRCA1_CNV#2_probe (SEQ ID NO: 15) | FAM-TGCTGAAGACCCCAAAGATC-BHQ1 |
| BRCA2_CNV#1_F (SEQ ID NO: 16) | TCCAAAGATTCAGAAAACTACTTTGA |
| BRCA2_CNV#1_R (SEQ ID NO: 17) | GAATGTGTGGCATGACTTGG |
| BRCA2_CNV#1_probe (SEQ ID NO: 18) | FAM-TGGAAGATGATGAACTGACAGA-BHQ1 |
| BRCA2_CNV#2_F (SEQ ID NO: 19) | CATTGATGGACATGGCTCTG |
| BRCA2_CNV#2_R (SEQ ID NO: 20) | TGAAAGGCAAAAATTCATCACA |
| BRCA2_CNV#2_probe (SEQ ID NO: 21) | FAM-CAAAAACAACTCCAATCAAGCA-BHQ1 |

Survival Analysis

Survival was analyzed by the Cox proportional-hazards regression method using clinical information and somatic mutation data of patients. After determining the hazard ratio (HR) and p-value of each mutation, Benjamini-Hochberg multiple testing correction was applied to address the risk of false positives because of multiple analysis (false discovery rate=0.05).

Protein-Protein Interaction Network and Gene Expression Analysis

STRING (database for interacting genes or proteins), KEGG (Kyoto Encyclopedia of Genes and Genomes), and DAVID (Database for Annotation, Visualization, and Integrated Discovery) were used to analyze oncogenic and tumor-suppression pathways in TNBC samples. In addition, CNV information, RNA expression (RNA-Seq), and mutation data of our TNBC samples were compared with those of TNBC samples from the TCGA database.

### Example 1: Exome Sequencing Using Samples from Breast Cancer Patients

Exome sequencing of the selected target genes was performed to discover genetic markers to develop companion diagnostic tests for prognosis and treatment of breast cancer patients.

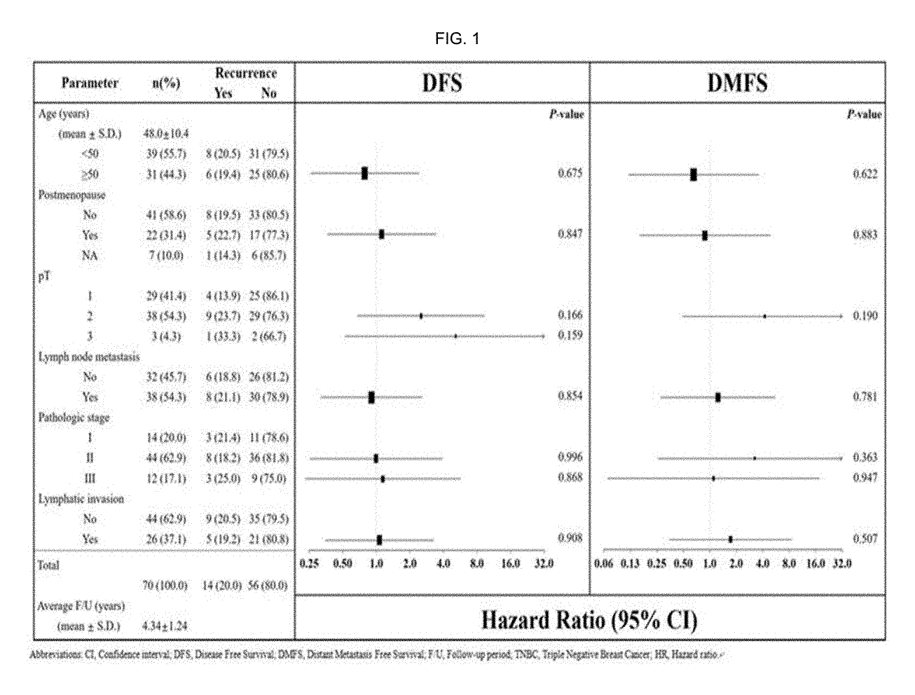

Tumor and matched adjacent normal tissues were collected from the total of 70 korean patients diagnosed with TNBC, and formalin-fixed, paraffin-embeded, followed by histological staining and analysis to confirm TNBC diagnosis. Basic clinicopathological characteristics of the patients included in this study and their impact on the hazard ratio are as described in FIG. 1. Part of the tissue samples were frozen immediately and used for genomic DNA extraction and exome sequencing. Somatic mutations occurred in the tumor tissues were identified by comparing and analyzing tumor and the surrounding normal tissues simultaneously. Only the genomic DNA samples with sufficient purity (2.1(260/280 ratio) 1.8; OD26/2301.5) were used for the analysis.

Library for the targeted exome sequencing was prepared using HaloPlex target selection panel. Next gene sequencing (NGS) was performed for the entire exome of the total of 368 target genes, which included 234 genes previously reported as cancer-associated and 134 transcription factor genes involved in cell growth. Genomic DNA was denatured and cut with 8 different restriction enzymes, followed by circularization with the biotinylated probes. Circularized target DNA fragments were sorted out using magnetic streptavidin beads, PCR-amplified, and subjected to library production for sequencing analysis using HiSeq2000. Among the reads generated during sequencing analysis, those with Phred Q score≥21 were mapped onto the standard human reference genome h19 using Burrows-Wheeler Alignment, and examined for single nucleotide variants (SNVs) and indels using Genome Analysis Toolkit and Samtools, and copy number variations (CNVs).

As a result, non-overlapping 292 somatic mutations (220 novel and 72 previously reported mutations) and 30 INDELs (7 novel and 2 previously reported insertions; 11 novel deletions and 10 previous reported deletions) were identified. Specifically, deletions were found in genes, such as WRN, PTPRD, ATM, GNAQ KIT, TCF4, CHUK, CTNNA1, EPHA5, TCF12, LIFR, PDGFRA, PLCG2, BUB1B, MLL2, RPS6KA2, and genes closely linked to breast cancers, such as BRCA1 and BRCA2. Exons of ATM gene harboring deletions are listed in Table 2.

TABLE 2
ATM gene exons found to have INDEL

| No. | Gene | Chr | Chr.Start | Chr.End | CDS | RNA [symbol] Accession | Length | Log2 [symbol] Ratio |
|---|---|---|---|---|---|---|---|---|
| 1 | ATM; + | chr11 | 108098343 | 108098777 | 1 | NM_000051.3 | 435 | −1.54 |
| 2 | ATM; + | chr11 | 108106341 | 108106664 | 4 | NM_000051.3 | 324 | −1.51 |
| 3 | ATM; + | chr11 | 108114570 | 108115002 | 5 | NM_000051.3 | 433 | −1.38 |
| 4 | ATM; + | chr11 | 108115557 | 108115651 | 6 | NM_000051.3 | 95 | −1.87 |
| 5 | ATM; + | chr11 | 108117552 | 108117970 | 7 | NM_000051.3 | 419 | −1.24 |
| 6 | ATM; + | chr11 | 108119635 | 108119861 | 8 | NM_000051.3 | 227 | −1.33 |
| 7 | ATM; + | chr11 | 108121358 | 108121895 | 9 | NM_000051.3 | 538 | −1.28 |
| 8 | ATM; + | chr11 | 108123491 | 108123760 | 11 | NM_000051.3 | 270 | −1.65 |
| 9 | ATM; + | chr11 | 108124401 | 108124774 | 12 | NM_000051.3 | 374 | −1.25 |
| 10 | ATM; + | chr11 | 108126905 | 108127039 | 13 | NM_000051.3 | 135 | −1.63 |
| 11 | ATM; + | chr11 | 108127056 | 108127150 | 13 | NM_000051.3 | 95 | −1.37 |
| 12 | ATM; + | chr11 | 108129654 | 108129761 | 15 | NM_000051.3 | 108 | −3.59 |
| 13 | ATM; + | chr11 | 108129763 | 108129904 | 15 | NM_000051.3 | 142 | −1.42 |
| 14 | ATM; + | chr11 | 108137818 | 108138104 | 16 | NM_000051.3 | 287 | −1.56 |
| 15 | ATM; + | chr11 | 108141712 | 108142276 | 18 | NM_000051.3 | 565 | −1.38 |
| 16 | ATM; + | chr11 | 108143210 | 108143319 | 20 | NM_000051.3 | 110 | −1.68 |
| 17 | ATM; + | chr11 | 108143334 | 108143661 | 20 | NM_000051.3 | 328 | −1.76 |
| 18 | ATM; + | chr11 | 108150201 | 108150482 | 22 | NM_000051.3 | 282 | −1.31 |
| 19 | ATM; + | chr11 | 108151619 | 108151968 | 23 | NM_000051.3 | 350 | −1.38 |
| 20 | ATM; + | chr11 | 108153356 | 108153661 | 24 | NM_000051.3 | 306 | −1.87 |
| 21 | ATM; + | chr11 | 108159648 | 108159873 | 27 | NM_000051.3 | 226 | −1.3 |
| 22 | ATM; + | chr11 | 108163165 | 108163547 | 29 | NM_000051.3 | 383 | −1.57 |
| 23 | ATM; + | chr11 | 108163943 | 108164346 | 30 | NM_000051.3 | 404 | −1.23 |
| 24 | ATM; + | chr11 | 108167887 | 108168120 | 32 | NM_000051.3 | 234 | −2.03 |
| 25 | ATM; + | chr11 | 108170327 | 108170711 | 33 | NM_000051.3 | 385 | −1.36 |
| 26 | ATM; + | chr11 | 108172287 | 108172581 | 34 | NM_000051.3 | 295 | −1.57 |
| 27 | ATM; + | chr11 | 108173476 | 108173939 | 35 | NM_000051.3 | 464 | −1.61 |
| 28 | ATM; + | chr11 | 108178598 | 108178757 | 37 | NM_000051.3 | 160 | −1.38 |
| 29 | ATM; + | chr11 | 108180936 | 108181065 | 38 | NM_000051.3 | 130 | −1.49 |
| 30 | ATM; + | chr11 | 108182987 | 108183335 | 39 | NM_000051.3 | 349 | −1.54 |
| 31 | ATM; + | chr11 | 108186368 | 108186773 | 40 | NM_000051.3 | 406 | −1.31 |
| 32 | ATM; + | chr11 | 108190619 | 108190836 | 43 | NM_000051.3 | 218 | −1.65 |
| 33 | ATM; + | chr11 | 108191840 | 108192243 | 44 | NM_000051.3 | 404 | −1.32 |
| 34 | ATM; + | chr11 | 108195973 | 108196348 | 45 | NM_000051.3 | 376 | −1.35 |
| 35 | ATM; + | chr11 | 108196542 | 108197155 | 46 | NM_000051.3 | 614 | −1.65 |
| 36 | ATM; + | chr11 | 108199597 | 108200065 | 48 | NM_000051.3 | 469 | −1.65 |
| 37 | ATM; + | chr11 | 108200817 | 108201257 | 49 | NM_000051.3 | 441 | −1.53 |
| 38 | ATM; + | chr11 | 108203436 | 108203635 | 52 | NM_000051.3 | 200 | −1.44 |
| 39 | ATM; + | chr11 | 108205637 | 108206070 | 54 | NM_000051.3 | 434 | −1.6 |
| 40 | ATM; + | chr11 | 108206544 | 108206782 | 55 | NM_000051.3 | 239 | −1.71 |
| 41 | ATM; + | chr11 | 108217803 | 108218254 | 58 | NM_000051.3 | 452 | −1.26 |
| 42 | ATM; + | chr11 | 108224379 | 108224745 | 59 | NM_000051.3 | 367 | −1.43 |
| 43 | ATM; + | chr11 | 108235683 | 108236008 | 61 | NM_000051.3 | 326 | −1.4 |

[symbol]

indicates data missing or illegible when filed

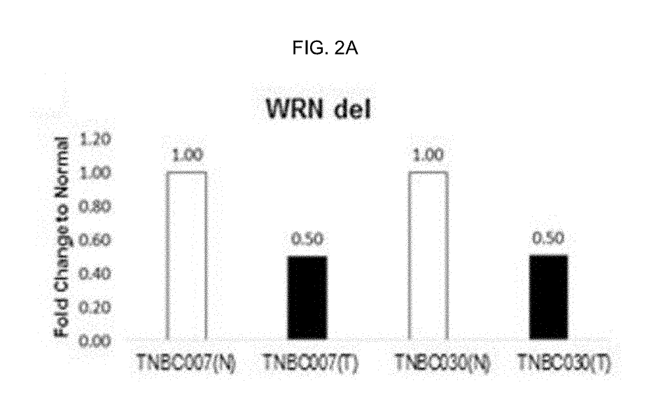

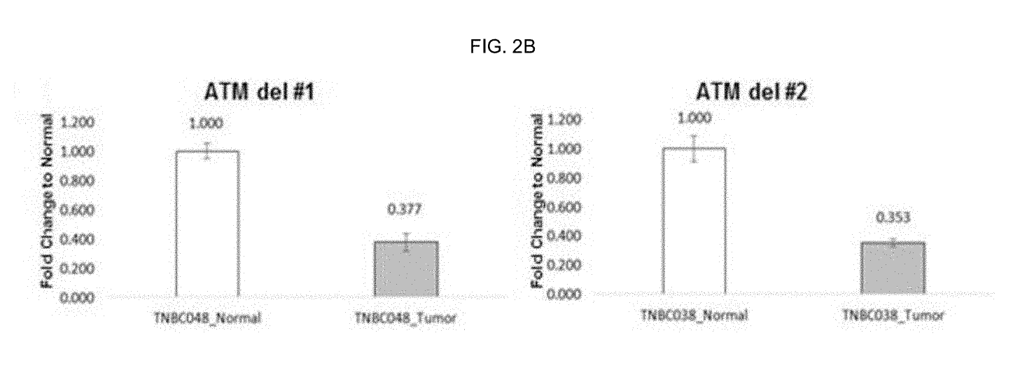

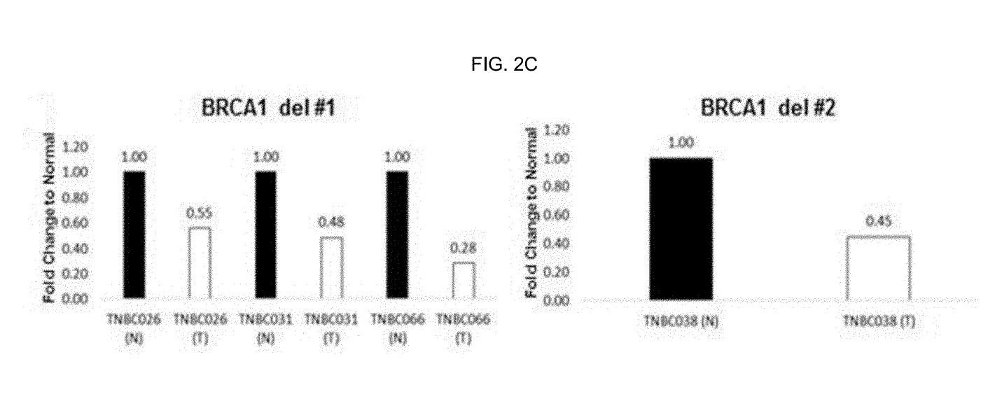

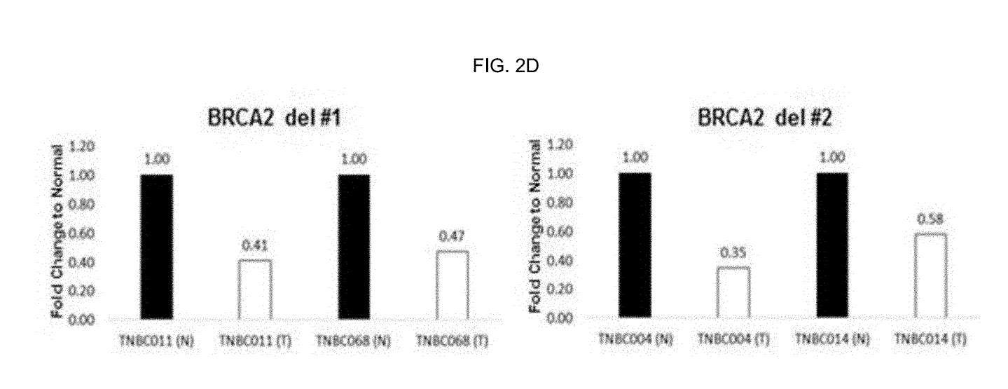

Furthermore, deletions in WRN and ATM were validated using qPCR among other genes found to have deletions by exome sequencing (FIGS. 2A-2D). Exons of the genes analyzed for deletions and corresponding patients are as described in Table 3. Samples of tumor and normal tissues were collected simultaneously from the patients identified with deletions by exome sequencing, and examined whether the gene indeed had deletions in the tumor tissues. qPCR analysis also included BRCA1 and BRCA2 genes which were confirmed to have germline mutations in many TNBC patients. Compared with TNBC patients without deletions, genomic DNA from TNBC patients identified with deletions had relatively low fold changes of those genes in the PCR products, suggesting reductions in copy numbers, which proves the existence of gene deletions.

TABLE 3
Exon region of the gene found to have deletions using qPCR

| Target | Samples | Gene | Exon | Chr | Accesion # | Chr. Start | Chr. End | Length |
|---|---|---|---|---|---|---|---|---|
| WRN del | TNBC007 | WRN | 6 | chr8 | NC_000008.10 | 30,924,479 | 30,924,555 | 77 |
| | TNBC030 | | | | | | | |
| ATM del #1 | TNBC048 | ATM | 16 | chr11 | NC_000011.9 | 108,129,659 | 108,129,758 | 100 |
| ATM del #2 | TNBC038 | ATM | 33 | chr11 | NC_000011.9 | 108,167,957 | 108,168,106 | 150 |
| BRCA1 del #1 | TNBC026 | BRCA1 | 6 | chr17 | NC_000017.10 | 41,256,098 | 41,256,247 | 150 |
| | TNBC031 | | | | | | | |
| | TNBC066 | | | | | | | |
| BRCA1 del #2 | TNBC038 | BRCA1 | 10 | chr17 | NC_000017.10 | 41,245,011 | 41,245,209 | 199 |
| BRCA2 del #1 | TNBC011 | BRCA2 | 11 | chr13 | NC_000013.10 | 32,915,138 | 32,915,247 | 110 |
| | TNBC068 | | | | | | | |
| BRCA2 del #2 | TNBC004 | BRCA2 | 14 | chr13 | NC_000013.10 | 32,929,298 | 32,929,465 | 168 |
| | TNBC014 | | | | | | | |

### Example 2: Analysis of Somatic Mutations and Copy Number Variations

Clinicopathological characteristics of 70 TNBC patients participated in the present study are as described in Table 4. During the follow-up period of 4.88 years on average, 21.4% (15/70) of the patients experienced recurrence, including 8 patients with distant metastases. It led to determine whether clinicopathological factors, such as age, primary tumor stage (pT), and lymph node metastasis, were associated with patient outcomes, such as disease-free survival (DFS) and distant metastasis-free survival (DMFS), however, no evidence supporting association between these factors and either DFS or DMFS was found.

Analysis showed 292 somatic single nucleotide variants (SNVs) and 30 somatic small insertions and deletions (INDELs) in 157 genes. Of these variants, 238 mutations were novel SNVs or INDELs that had not been reported previously in either the COSMIC or dbSNP database (FIG. 2A). Frequently mutated genes and types of somatic mutations are as listed in Table 5 and Table 6.

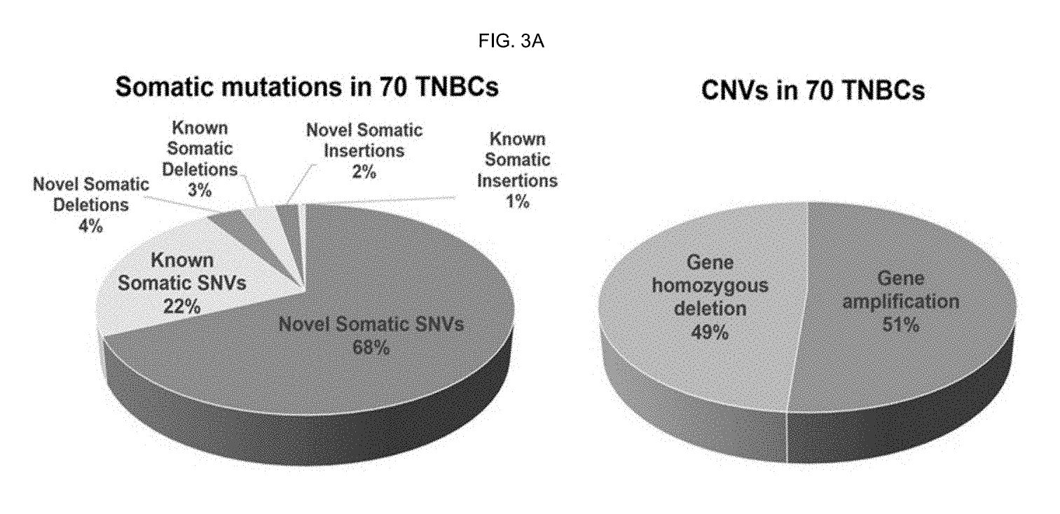

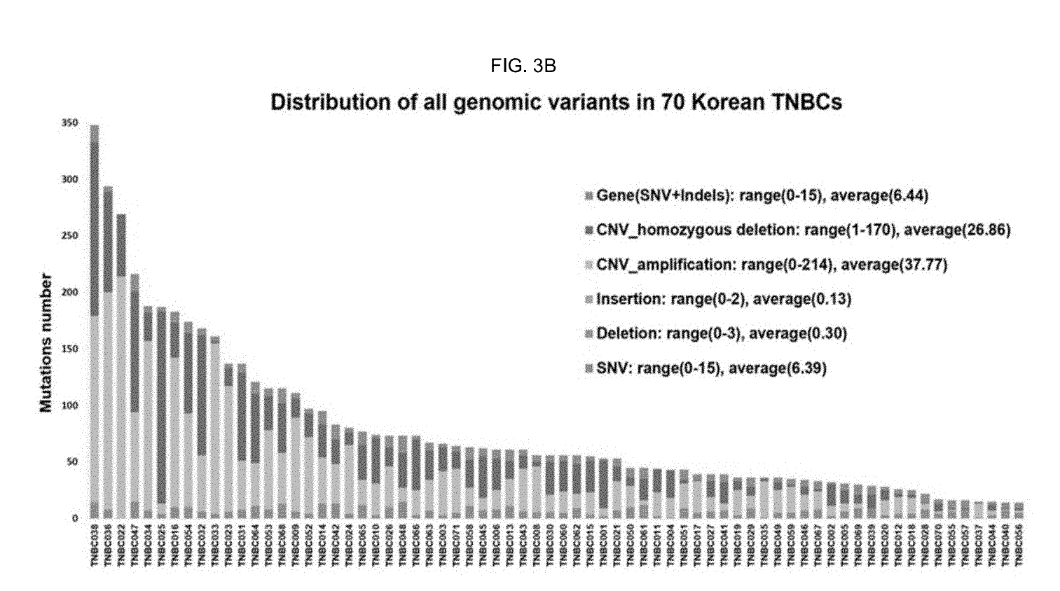

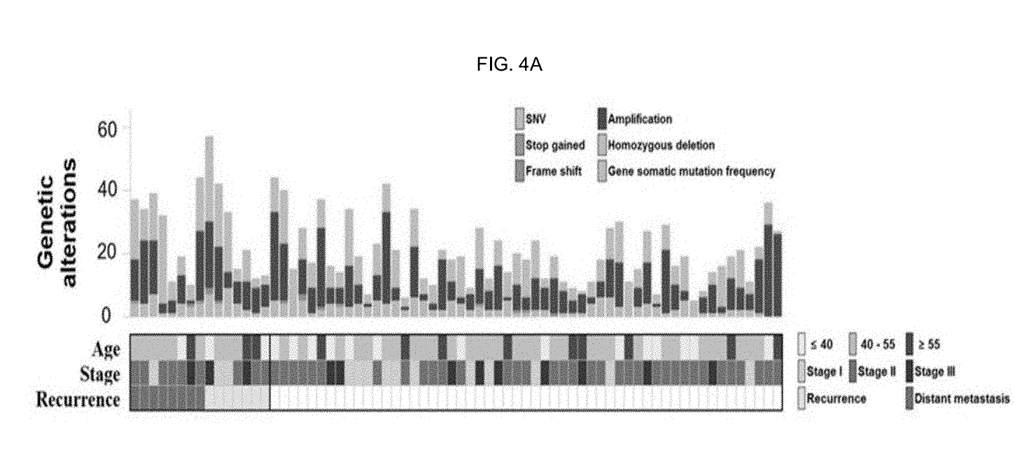

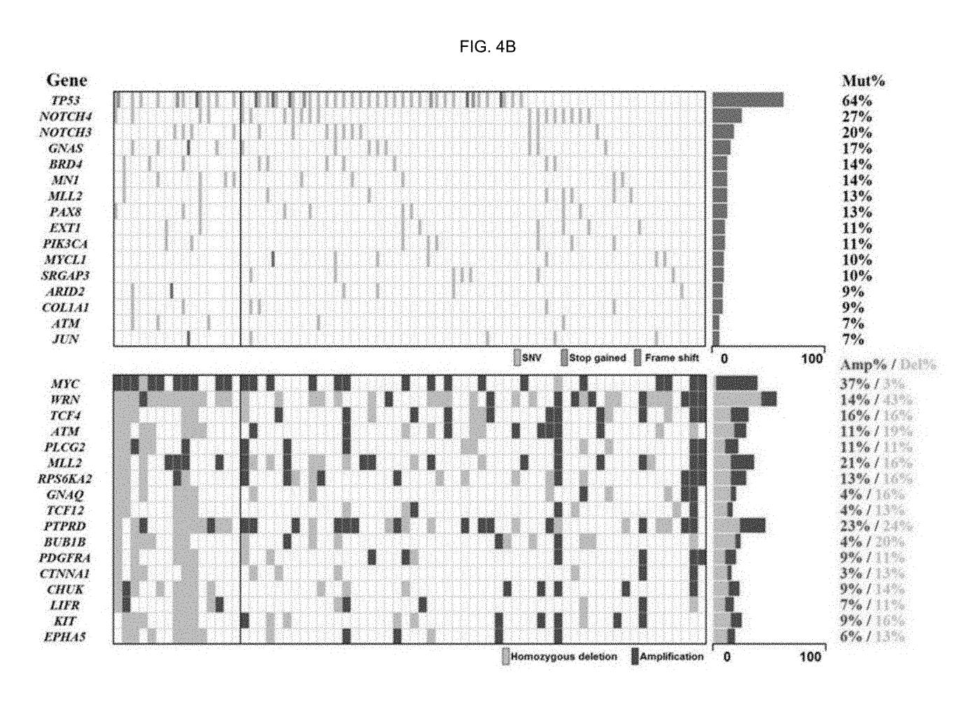

In addition, 5 (7%, 5/70) of the somatic mutations in TP53 were stop-gain mutations, 6 (9%, 6/70) were frameshifts. Frameshift mutations were also detected in four other gene, GNAS, ARID2, JUN and MYCL1 (FIGS. 3A-3B). Sanger capillary sequencing detected two somatic mutations in TP53 (c.637C>T, c.578A>G) as well.

Copy number variation (CNV) analysis identified an average of 37.77 (range, 0-214) amplified genes and 26.86 (range, 1-170) homozygously deleted genes per patient (FIG. 2B). Genes with frequent CNV amplifications and homozygous deletions are as listed in Table 5. Homozygous deletions of TP53, a tumor suppressor gene with the highest mutation frequency in the present study, were observed in 10 other TNBC patients, indicating that 55 (79%) of the 70 patients in the present study cohort had either mutated or deleted TP53. In addition to the deleterious germline mutations described previously, somatic homozygous deletions of BRCA1 and BRCA2 were observed in the genomes of 12 and 10 patients, respectively (Table 5). Some of these homozygous deletions were restricted to a single exon, whereas others encompassed several exons.

TABLE 4
Clinical pathologic features of TNBC patients

| Parameter | n(%) | |
|---|---|---|
| Age | | |
| (mean ± S.D.) | 48.0 ± 10.4 | |
| <50 | 39 | (55.7) |
| ≥50 | 31 | (44.3) |
| Postmenopause | | |
| No | 41 | (58.6) |
| Yes | 22 | (31.4) |
| NA | 7 | (10.0) |
| pT | | |
| 1 | 29 | (41.4) |
| 2 | 38 | (54.3) |
| 3 | 3 | (4.3) |
| Lymph node metastasis | | |
| No | 32 | (45.7) |
| Yes | 38 | (54.3) |
| Pathologic stage | | |
| I | 14 | (20.0) |
| II | 44 | (62.9) |
| III | 12 | (17.1) |
| Lymphatic invasion | | |
| No | 44 | (62.9) |
| Yes | 26 | (37.1) |
| Recurrence | | |
| No | 55 | (78.6) |
| Yes | 15 | (21.4) |
| Total | | |
| | 70 | (100.0) |
| Average F/U | | |

| | |
|---|---|
| (mean ± S.D.) | 4.88 ± 1.34 |

Abbreviations: pT, Primary tumor stage;

TABLE 5
List of frequently mutated genes

| Somatic Mutation | | | Amplified | | Homozygous | |
|---|---|---|---|---|---|---|
| Genes | | | Genes | | Deleted Genes | |
| | Fre- | | | Fre- | | Fre- |
| | quen- | | | quen- | | quen- |
| Gene | cy (%) | | Gene | cy (%) | Gene | cy (%) |
| TP53 | 45 | (64) | NDRG1 | 36 (51) | WRN | 30 (43) |
| NOTCH4 | 19 | (27) | UBR5 | 32 (46) | IL6ST | 22 (31) |
| NOTCH3 | 14 | (20) | PTK2 | 32 (46) | APC | 21 (30) |
| GNAS | 12 | (17) | RECQL4 | 26 (37) | PTK1B | 20 (29) |
| BRD4 | 10 | (14) | MYC | 26 (37) | NF1 | 19 (27) |
| MN1 | 10 | (14) | IKBKE | 25 (36) | SETD2 | 18 (26) |
| MLL2 | 9 | (13 | EXT1 | 25 (36) | PTPRD | 17 (24) |
| PAX8 | 9 | (13) | CDK2 | 24 (34) | PBRMI | 17 (24) |
| EXT1 | 8 | (11) | NTRK1 | 24 (34) | MLL3 | 16 (23) |
| PIK3CA | 8 | (11) | DDR2 | 22 (31) | PCM1 | 16 (23) |
| ETV4 | 7 | (10) | MCLI | 22 (31) | PLD2 | 15 (21) |
| GLI3 | 7 | (10) | TPR | 20 (29) | PIK3RI | 15 (21) |
| HOOK3 | 7 | (10) | PARP1 | 19 (27) | CDK2 | 14 (20) |
| MYCL1 | 7 | (10) | TPM3 | 19 (27) | CSF1R | 14 (20) |
| SRGAP3 | 7 | (10) | PRCC | 19 (27) | BUB1B | 14 (20) |
| ARID2 | 6 | (9) | RNF213 | 19 (27) | CDK12 | 14 (20) |
| COLIA1 | 6 | (9) | ERC1 | 19 (27) | MTOR | 13 (19) |
| MTOR | 6 | (9) | FH | 18 (26) | CHEK2 | 13 (19) |
| TRIM62 | 6 | (9) | NBN | 18 (26) | ATM | 13 (19) |
| ATM | 5 | (7) | RGL1 | 17 (24) | RBI | 13 (19) |
| BAPI | 5 | (7) | PTPRD | 16 (23) | MAP3K1 | 13 (19) |
| JUN | 5 | (7) | TIAM1 | 16 (23) | TLAM1 | 12 (17) |
| KDM5C | 5 | (7) | NOTCH4 | 16 (23) | ERCC2 | 12 (17) |
| PPP2R1A | 5 | (7) | IGF1R | 16 (23) | KTN1 | 12 (17) |
| BRCA2 | 4 | (6) | IKBKB | 16 (23) | BRCA1 | 12 (17) |
| CDKN2A | 4 | (6) | GATA3 | 16 (23) | TSHR | 12 (17) |
| FGFR3 | 4 | (6) | PBX1 | 16 (23) | MLL2 | 11 (16) |
| GRIN2D | 4 | (6) | MLL2 | 15 (21) | PRKDC | 11 (16) |
| MAP3K1 | 4 | (6) | FLT4 | 15 (21) | TCF4 | 11 (16) |
| MAPK8IP3 | 4 | (6) | EGFR | 15 (21) | USP6 | 11 (16) |
| PIK3R1 | 4 | (6) | RPTOR | 15 (21) | BPS6KA2 | 11 (16) |
| PTCH1 | 4 | (6) | RUNXIT1 | 15 (21) | TAF1 | 11 (16) |
| RPTOR | 4 | (6) | COX6C | 15 (21) | KIT | 11 (16) |
| SFPQ | 4 | (6) | FLNA | 14 (20) | MAP2K2 | 11 (16) |
| AKAP9 | 3 | (4) | TSC2 | 14 (20) | EML4 | 11 (16) |
| ATRX | 3 | (4) | ATR | 14 (20) | RPS6KA3 | 11 (16) |
| BAX | 3 | (4) | MAML2 | 14 (20) | GNAQ | 11 (16) |
| BRD3 | 3 | (4) | NTRK3 | 14 (20) | KIAA1549 | 10 (14) |
| CD74 | 3 | (4) | CRTC3 | 14 (20) | PMS1 | 10 (14) |
| CDKN1A | 3 | (4) | TFEB | 14 (20) | BRCA2 | 10 (14) |
| CIC | 3 | (4) | MLL3 | 13 (19) | CHUK | 10 (14) |
| EGFR | 3 | (4) | ERCC2 | 13 (19) | ALDH2 | 10 (14) |
| EPHA5 | 3 | (4) | SMARCA4 | 13 (19) | FGFR3 | 10 (14) |
| FLNA | 3 | (4) | EP300 | 13 (19) | TP53 | 10 (14) |

TABLE 6
High frequency somatic mutation

| | | | | | | | Mutation Assessment | |
|---|---|---|---|---|---|---|---|---|
| | | | | | | | | PolyPhen2 |
| | Nucleotide | Amino Acid | Frequency | | Somatic | Previously | SIFT | HDIV |
| Gene Name | Change | Change | (%) | | mutation type | reported | score | score |
| NOTCH4 | c.625T > G | p.T209P | 9 | (13) | Heterozygous | Novel | 0.01 | 0.9900 |
| | | | | | | | | 0.9030 |
| ETV4 | c.770T > G | p.V257G | 7 | (10) | Heterozygous | Novel | 0.11 | 0.8720 |
| | | | | | | | | 0.9420 |
| EXT1 | c.148T > G | p.S50R | 7 | (10) | Heterozygous | Novel | 0.74 | 0.0000 |
| GNAS | c.1264T > C | p.S422P | 7 | (10) | Heterozygous | Novel | 0.18 | 0.0030 |
| NOTCH3 | c.6841C > G | p.A2281P | 7 | (10) | Heterozygous | Novel | 0.86 | 0.8380 |
| COL [symbol] A [symbol] | c.3746T > C | p.E1249G | 6 | (9) | Heterozygous | Novel | 0.00 | 0.7700 |
| MLL2 | c.2482G > C | p.P828A | 6 | (9) | Heterozygous | Novel | 0.00 | 0.0080 |
| TP53 | c.1103A > C | p.H368P | 6 | (9) | Heterozygous | Novel | 0.21 | 0.0000 |
| ARID2 | c.3803A > C | p.N1268T | 5 | (7) | Heterozygous | Novel | 0.00 | 0.0540 |
| | | | | | | | | 0.0310 |
| | | | | | | | | 0.0320 |
| NOTCH4 | c.1118T > G | p.T40P | 5 | (7) | Heterozygous | Novel | 0.03 | 0.0320 |
| | | | | | | | | 0.2470 |
| BRD4 | c.2470T > G | p.T824P | 4 | (6) | Heterozygous | Novel | 0.12 | 0.0000 |
| GLI3 | c.2687T > G | p.D896A | 4 | (6) | Heterozygous | Novel | 0.00 | 1.0000 |
| HOOK3 | c.62A > C | p.Q21P | 4 | (6) | Heterozygous | Novel | 0.07 | 1.0000 |
| | | | | | | | | 0.9980 |
| MN [symbol] | c.2780G > A | p.T927R | 4 | (6) | Heterozygous | Novel | 0.15 | 1.0000 |
| MTOR | c.5480T > G | p.N1827T | 4 | (6) | Heterozygous | Novel | 0.46 | 0.0000 |
| NOTCH4 | c.3064C > G | p.A1022P | 4 | (6) | Heterozygous | Novel | NA | 0.9810 |
| PAX8 | c.695A > C | p.H232P | 4 | (6) | Heterozygous | Novel | 0.02 | 0.0010 |
| | | | | | | | | 0.0000 |
| PPP2R1A | c.584T > G | p.V195G | 4 | (6) | Heterozygous | Novel | 0.07 | 0.9680 |
| | | | | | | | | 0.5880 |
| TRIM62 | c.1094T > G | p.I365S | 4 | (6) | Heterozygous | Novel | 0.00 | 1.0000 |
| ATM | c.6337A > C | p.T2113P | 3 | (4) | Heterozygous | Novel | 0.28 | 0.0010 |
| BAP1 | c.626T > G | p.V209G | 3 | (4) | Heterozygous | Novel | 0.00 | 1.0000 |
| CD74 | c.455T > G | p.L152R | 3 | (4) | Heterozygous | Novel | 1.00 | 0.8170 |
| | | | | | | | | 0.0910 |
| | | | | | | | | 0.0150 |
| CDKN [symbol] A | c.93C > A | p.S31R | 3 | (4) | Homozygous | dbSNP | 0.99 | 0.0000 |
| KDM5C | c2254A > C | p.T752P | 3 | (4) | Heterozygous | Novel | 0.17 | 0.0850 |
| | | | | | | | | 0.1990 |
| MAP3K14 | c2024A > C | p.H675P | 3 | (4) | Heterozygous | Novel | 0.00 | 0.0000 |
| MAPK8IP3 | c.763T > C | p.S255P | 3 | (4) | Heterozygous | Novel | 0.01 | 0.0100 |
| | | | | | | | | 0.7250 |
| | | | | | | | | 0.9840 |
| MCL1 | c.116A > G | p.E39G | 3 | (4) | Heterozygous | Novel | NA | NA |
| | | | | | | | 0.54 | 0.0000 |
| MN [symbol] | c.2773G > A | p.E925K | 3 | (4) | Heterozygous | Novel | 0.29 | 0.9650 |
| NOTCH3 | c.6865G > C | p.A2289P | 3 | (4) | Heterozygous | dbSNP | 0.37 | 0.0000 |
| PAX8 | c.665A > C | p.H222P | 3 | (4) | Heterozygous | Novel | 0.12 | 0.5310 |
| | | | | | | | | 0.0030 |
| | | | | | | | | 0.3960 |
| | | | | | | | | 0.2010 |
| | | | | | | | | 0.1250 |
| PAX8 | c.734T > G | p.Y245S | 3 | (4) | Heterozygous | Novel | 0.03 | 0.6480 |
| | | | | | | | | 0.6390 |
| | | | | | | | | 0.0040 |
| | | | | | | | | 0.2720 |
| | | | | | | | | 0.7170 |
| PIK3CA | c.3140A > G | p.H1047R | 3 | (4) | Heterozygous | dbSNP | 0.16 | 0.6390 |
| | | | | | | COSMIC | | |
| PIK3CA | c.821G > A | p.R274K | 3 | (4) | Heterozygous | dbSNP | 0.03 | 0.9790 |
| PIK3R1 | c.367G > C | p.A123P | 3 | (4) | Heterozygous | Novel | 0.21 | 0.3380 |
| RPTOR | c.2557A > C | P.T853P | 3 | (4) | Heterozygous | Novel | 0.29 | 0.2550 |
| | | | | | | | | 0.1670 |
| SRGAP3 | c.3116T > C | p.F1039S | 3 | (4) | Heterozygous | Novel | 0.26 | 0.2550 |
| | | | | | | | | 0.1650 |
| TP53 | c.821G > T | p.R273L | 3 | (4) | Heterozygous | dbSNP | 0.00 | 0.9990 |
| | | | | | | COSMIC | | 1.0000 |
| TP53 | c.746G > A | p.R248Q | 3 | (4) | Homozygous | dbSNP | 0.01 | 1.0000 |
| | | | | | Heterozygous | COSMIC | | 0.9940 |

| | Mutation Assessment | | | | | |
|---|---|---|---|---|---|---|
| | PolyPhen2 | | | | Mutation | Mutation |
| | HDIV | HVAR | HVAR | LRT | Taster | Assessor |
| Gene Name | pred | score | pred | score | score | score |
| NOTCH4 | D | 0.9270 | D | 0.1942 | 0.7871 | 1.3850 |
| | P | 0.6030 | P | | | |
| ETV4 | P | 0.8260 | P | 0.0134 | 0.8814 | 1.9950 |
| | | 0.8120 | | | | |
| EXT1 | B | 0.0000 | B | 0.0025 | 0.3789 | 0.0000 |
| GNAS | B | 0.0020 | B | 0.0000 | 0.0000 | 1.5250 |
| NOTCH3 | P | 0.2020 | B | NA | 0.5542 | 0.0000 |
| COL1A1 | P | 0.3270 | B | 0.0000 | 0.7868 | 3.4800 |
| MLL2 | B | 0.0060 | B | NA | NA | 0.5500 |
| TP53 | B | 0.0010 | B | 0.4522 | 0.0857 | 0.3450 |
| ARID2 | B | 0.0270 | B | 0.0000 | 0.9744 | 0.9750 |
| | | 0.0210 | | | | |
| NOTCH4 | B | 0.0150 | B | 0.1892 | 0.9635 | 2.5850 |
| | | 0.0870 | | | | |
| BRD4 | B | 0.0000 | B | 0.1482 | 0.0008 | −0.6900 |
| GLI3 | D | 1.0000 | D | 0.0000 | 1.0000 | 2.8350 |
| HOOK3 | D | 0.9980 | D | 0.0000 | 0.8988 | 2.4150 |
| | | 1.0000 | | | | |
| MNI | D | 0.9980 | D | 0.0000 | 0.9374 | 0.8050 |
| MTOR | B | 0.0010 | B | 0.0234 | 0.0171 | 0.3450 |
| NOTCH4 | D | 0.6030 | P | 0.0106 | 0.8376 | 0.5500 |
| PAX8 | B | 0.0010 | B | 0.2301 | 0.0635 | 0.2050 |
| | | 0.0020 | | | | |
| | | 0.0000 | | | | |
| PPP2R1A | D | 0.3140 | B | 0.0000 | 1.0000 | 2.9600 |
| | P | 0.2850 | | | | |
| TRIM62 | D | 0.9980 | D | 0.0000 | 0.9990 | 2.4750 |
| ATM | B | 0.0040 | B | 0.6501 | 0.0022 | 0.0000 |
| BAP1 | D | 0.9980 | D | 0.0000 | 1.0000 | 3.5250 |
| CD74 | P | 0.4990 | P | 0.7923 | 0.0338 | 1.1000 |
| | B | 0.0270 | B | | | |
| | | 0.0380 | | | | |
| | | 0.2840 | | | | |
| CDKNIA | B | 0.0010 | B | 0.9321 | 0.0024 | −0.1300 |
| KDM5C | B | 0.0800 | B | 0.0000 | 0.7922 | 1.9150 |
| | | 0.1730 | | | | |
| MAP3K14 | 0 | 0.0000 | 0 | 0.0000 | 0.0000 | 0.0000 |
| MAPK8IP3 | B | 0.0190 | B | 0.0002 | 0.9997 | 1.8950 |
| | P | 0.3270 | P | | | |
| | D | 0.6420 | | | | |
| MCL1 | | NA | | NA | NA | −0.5500 |
| | B | 0.0010 | B | 0.0000 | 0.0005 | |
| | | 0.0000 | | | | |
| MNI | D | 0.6550 | P | 0.0000 | 0.4251 | 0.5500 |
| NOTCH3 | B | 0.0000 | B | NA | 0.5542 | 0.0000 |
| PAX8 | P | 0.0990 | B | 0.0014 | 0.6003 | 1.5450 |
| | B | 0.0020 | | | | |
| | | 0.0680 | | | | |
| | | 0.3000 | | | | |
| | | 0.0840 | | | | |
| PAX8 | P | 0.4260 | B | 0.0168 | 0.2135 | 1.8800 |
| | B | 0.1100 | | | | |
| | | 0.0010 | | | | |
| | | 0.3280 | | | | |
| | | 0.2430 | | | | |
| PIK3CA | P | 0.0850 | B | 0.0000 | 0.9999 | 0.0000 |
| PIK3CA | D | 0.8920 | P | 0.0000 | 0.9997 | 2.1750 |
| PIK3R1 | B | 0.2000 | B | 0.0006 | 0.8999 | 1.3550 |
| RPTOR | B | 0.1010 | B | 0.0001 | 0.4881 | 1.5900 |
| | | 0.0460 | | | | |
| SRGAP3 | B | 0.0700 | B | 0.0000 | 0.8194 | 1.7500 |
| | | 0.0320 | | | | |
| TP53 | D | 0.9860 | D | 0.0000 | 1.0000 | 3.1450 |
| | | 0.9900 | | | | |
| | | 0.9870 | | | | |
| | | 0.9880 | | | | |
| TP53 | D | 0.9960 | D | 0.0000 | 1.0000 | 2.9700 |
| | | 0.8820 | P | | | |
| | | 0.9990 | | | | |
| | | 0.9950 | | | | |

Abbreviations: B, Benign; P, Possibly damaging; D, Probably damaging. [symbol]
indicates data missing or illegible when filed

### Example 3: Correlation Between Homozygote Deletions and the Survival Rate of Breast Cancer Patients

It was determined whether homozygous deletions identified by targeted exome sequencing were associated with the prognosis of breast cancer patients.

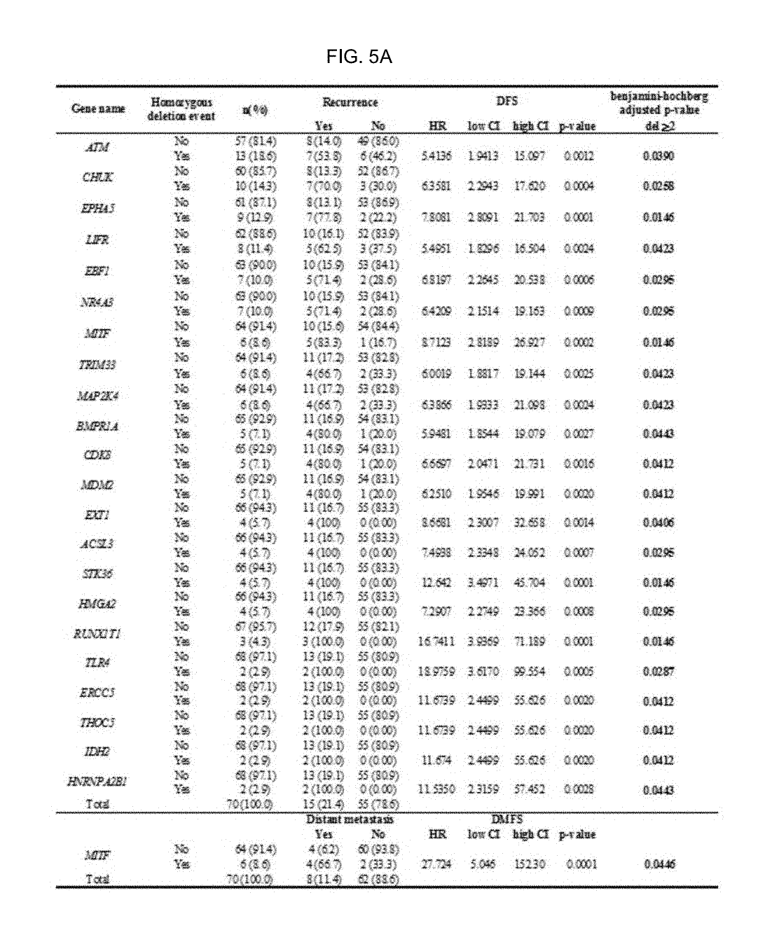

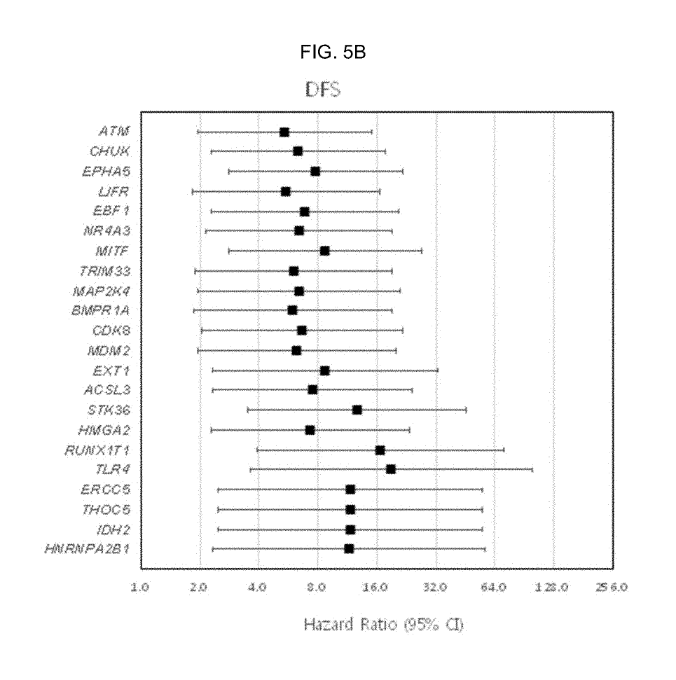

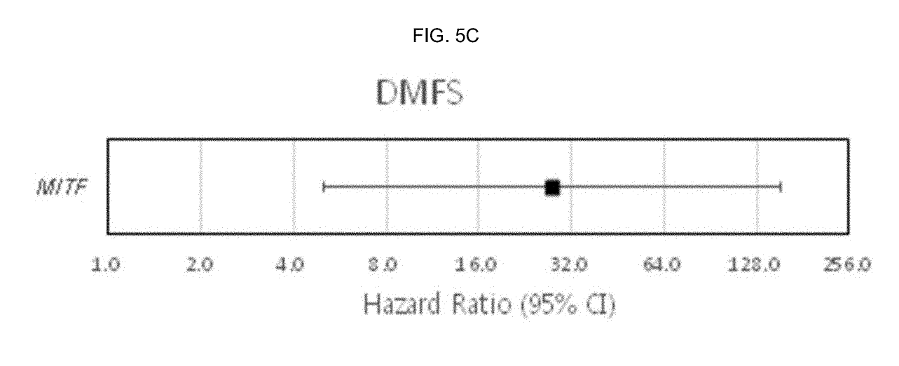

For all the TNBC patients included in the present study, correlation between homozygous deletions in genes such as ATM, CHUK, EPHA5, LIFR, EBF1, NR4A3, MITF, TRIM33, MAP2K4, BMPR1A, CDK8, MDM2, EXT1, ACSL3, STK36, HMGA2, RUNX1T1, TLR4, ERCC5, THOC5, IDH2, and HNRNPA2B1, and proportional hazard ratio was analyzed using parameters such as recurrence and distance metastasis (FIGS. 5A-5C). Particularly, homozygous deletions in genes such as ATM (recurrence, HR=5.4136, p-value=0.0012), CHUK (recurrence, HR=6.3581, p-value=0.0004), EPHA5 (recurrence, HR=7.8081, p-value=0.0001), LIFR (recurrence, HR=5.4951, p-value=0.0024), MITF (distant metastasis, HR=27.724, p-value=0.0001) were associated with an increased risk of recurrence or distant metastases in patients with TNBC.

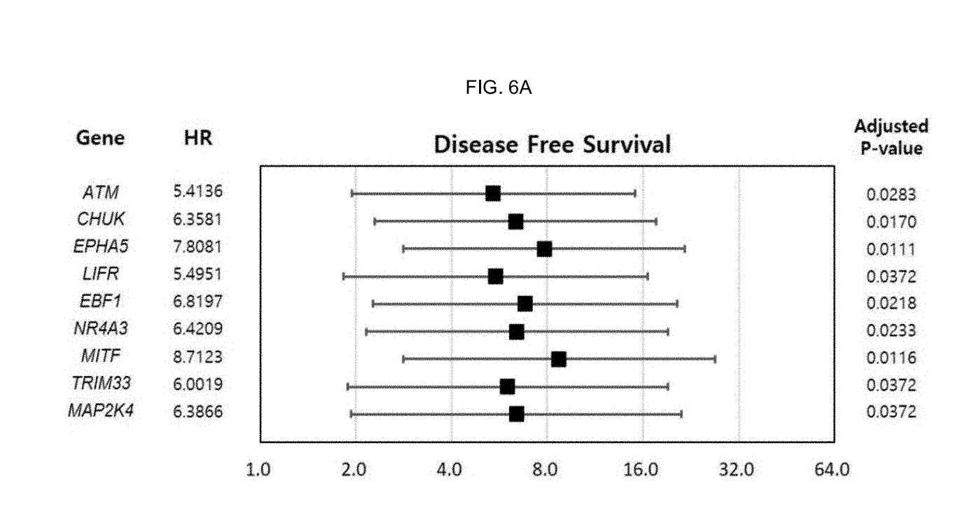

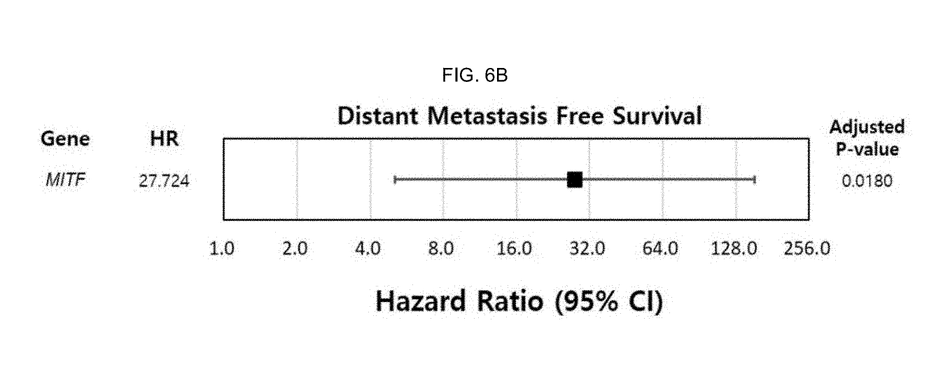

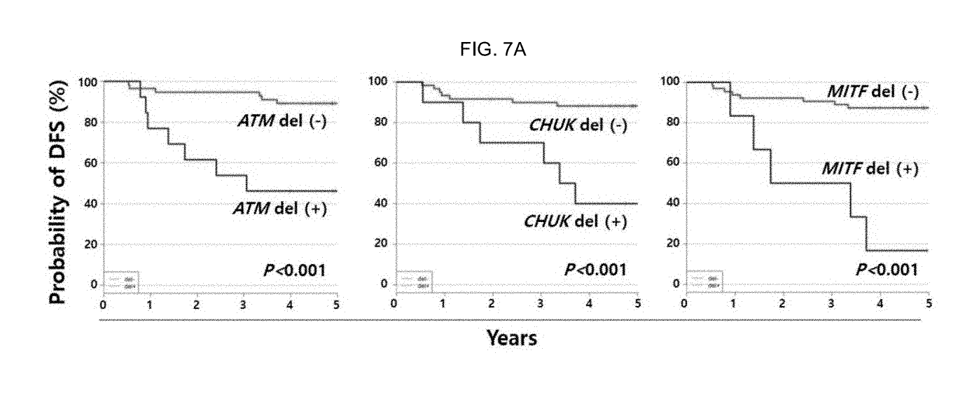

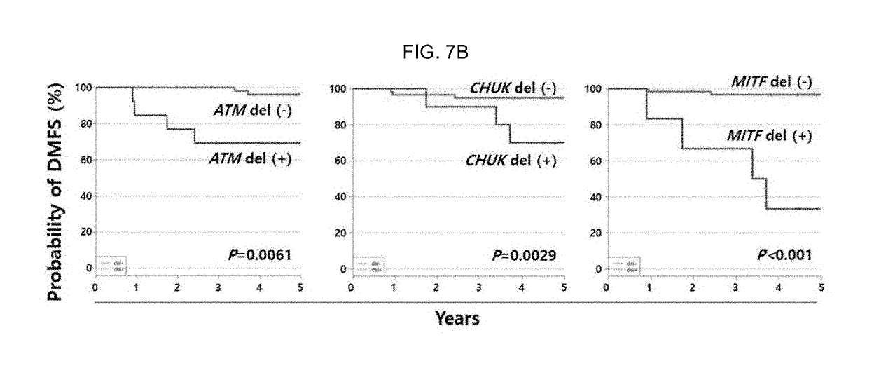

In addition, correlation between homozygous deletions in the above-mentioned genes and disease free survival (DFS) or distant metastasis free survival (DMFS) was analyzed (FIGS. 6A-6B). The result showed that homozygous deletions in genes such as ATM, CHUK, EPHA5, LIFR, EBF1, NR4A3, MITF, TRIM33, and MAP2K4 had significant impact on survival rate of patients with TNBC. Furthermore, Kaplan-Meier analysis (FIGS. 7A-7B) revealed that survival time was shorter in patients with deletions in genes such as ATM, CHUK and MITF compared with patients without such deletions, suggesting a poor prognosis. These results prove homozygous deletions in these genes are inversely correlated with the prognosis of TNBC patients.

### Example 4: TCGA Data Analysis

Associations between levels of mRNA expression and copy number alteration of genes identified as frequently amplified in the 70 Korean TNBC samples were analyzed using CNV and mRNA expression data from The Cancer Genome Atlas (TCGA) breast cancer database.

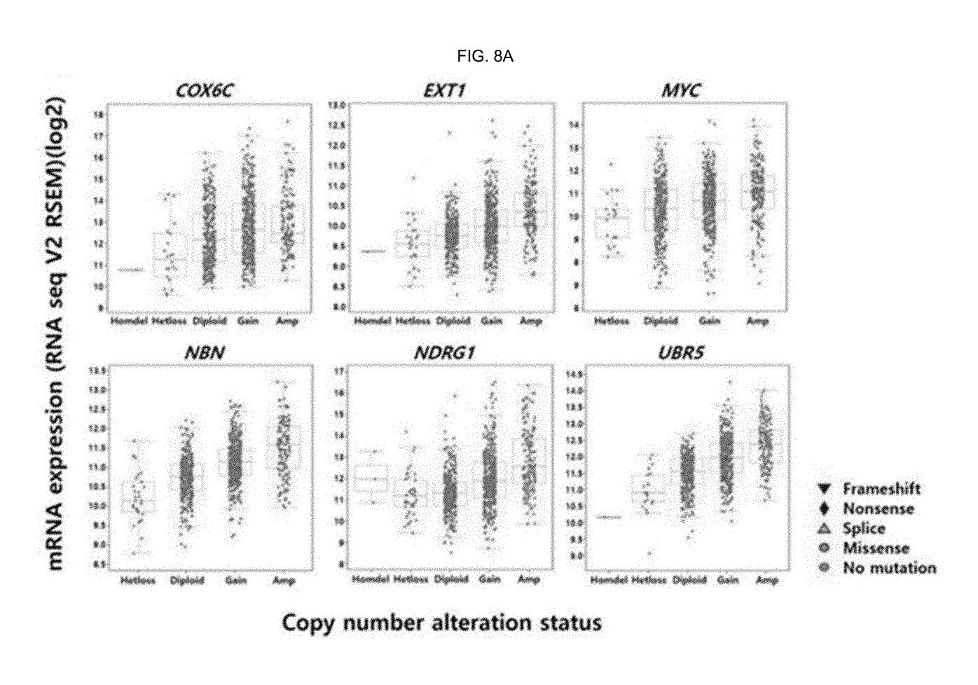

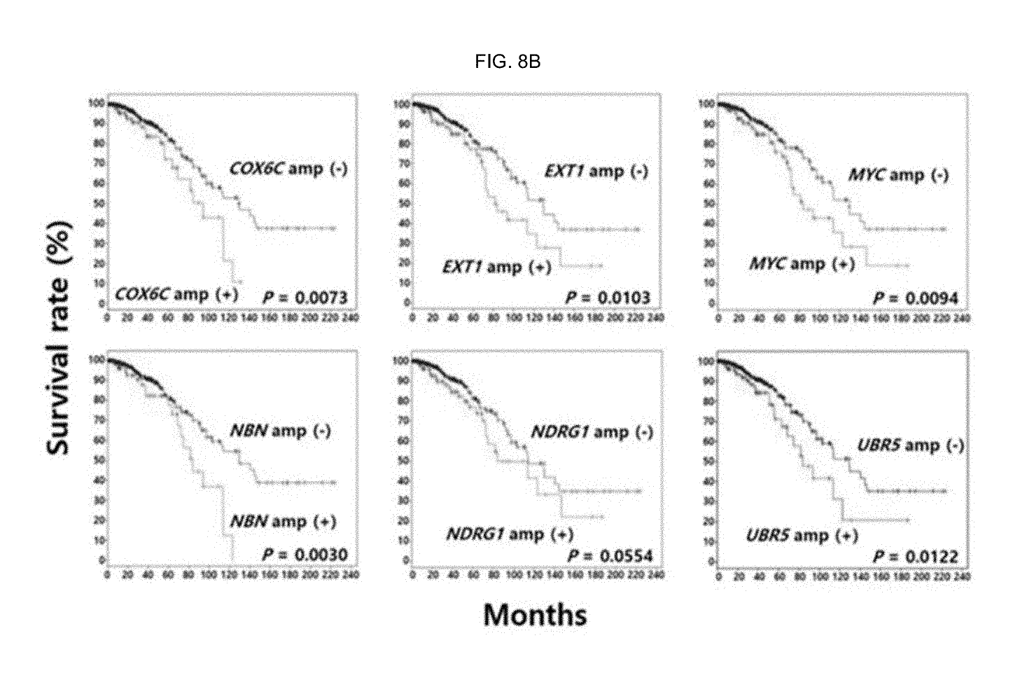

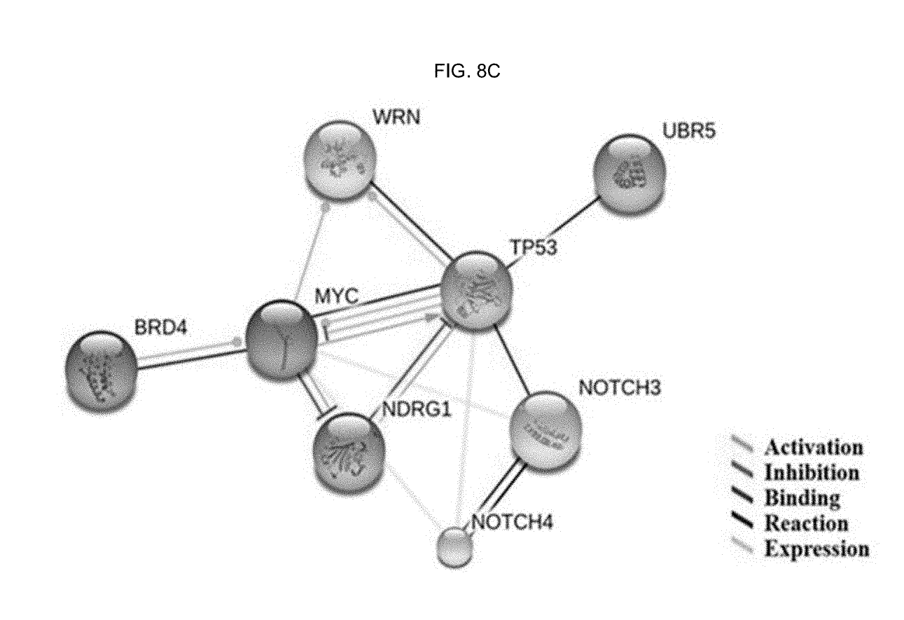

We found that copy number gain or amplification of six genes (NDRG1, UBR5, MYC, EXT1, NBN, and COX6C) was positively correlated with high mRNA expression (FIG. 8A). Kaplan-Meier analysis showed that the overall survival rates were significantly lower in breast cancer patients with than without amplification of one of these genes (log rank test; NDRG1, P=0.0554; UBR5, P=0.0122; MYC, P=0.0094; EXT1, P=0.0103; NBN, P=0.0030; and COX6C, P=0.0073) (FIG. 8B).

Next, using STRING (Search Tool for the Retrieval of Interacting Genes/Proteins, v.10), network interaction was analyzed for proteins encoded by genes with the most frequent genetic alterations (i.e., somatic non-synonymous mutations and CNVs) in the cohort of 70 Korean patients with TNBC. It was found that DNA damage response genes, such as TP53 and WRN, were frequently mutated in our TNBC cohort (FIG. 8C). Notably, mutual exclusivity analysis using 500 clinical breast cancer samples from the TCGA database indicated a high likelihood of co-occurrence of alterations in the TP53, MYC, WRN, NDRG1, NOTCH3, UBR5, and BRD4 genes, all of which are involved in the above-mentioned interaction network.

### INDUSTRIAL APPLICABILITY

As described above, the methods and compositions of the present invention for detecting the deletion of multiple genes as markers can be used to develop markers for determining the prognosis of breast cancer, particularly triple negative breast cancer patients.

1. A method for detecting a marker of a prognosis of a breast cancer patient, the method comprising;
obtaining a sample of a test subject;
extracting genomic DNA from the sample;
confirming the presence or absence of the deletion of a gene in the extracted genomic DNA; and
determining that the test subject has a breast cancer with a poor prognosis in case the presence of the deletion of a gene is confirmed in the genomic DNA.

2. The method of claim 1, wherein the gene is at least one gene selected from the group consisting of ATM, CHUK, EPHA5, LIFR, EBF1, NR4A3, MITF, TRIM33, MAP2K4, BMPR1A, CDK8, MDM2, EXT1, ACSL3, STK36, HMGA2, RUNX1T1, TLR4, ERCC5, THOC5, IDH2 and HNRNPA2B1.

3. The method of claim 1, wherein the deletion of the gene is a homozygous deletion of the gene.

4. The method of claim 1, wherein the presence or absence of the deletion of a gene is confirmed by a method selected from the group consisting of direct sequencing, next generation sequencing, targeted exome sequencing, sequencing read depth method, whole genome sequence assembly, quantitative PCR, multiplex amplifiable probe hybridization (MAPH), multiplex ligation-dependent probe amplification (MLPA), paralogue ratio test (PRT), array comparative genomic hybridization (array CGH), SNP microarray, fiber FISH, southern blotting and pulsed field gel electrophoresis (PFGE).

5. The method of claim 1, wherein the sample of the test subject is a breast cancer tissue.

6. The method of claim 1, wherein the breast cancer is a triple negative breast cancer.

7. The method of claim 6, wherein the triple negative breast cancer is determined by confirming the absence of the gene expression of estrogen receptor, progesterone receptor and HER2 in the breast cancer tissue of the test subject, respectively.

8. The method of claim 7, wherein the absence of the gene expression is determined by the absence of mRNA or protein of the gene.

9. The method of claim 5, wherein the sample of the test subject further comprises a normal tissue obtained from the same test subject.

10. The method of claim 9, wherein the normal tissue sample is in the absence of the deletion of a gene.

11. A composition comprising an agent capable of confirming the deletion of a gene.

12. The composition of claim 11, wherein the gene is at least one gene selected from the group consisting of ATM, CHUK, EPHA5, LIFR, EBF1, NR4A3, MITF, TRIM33, MAP2K4, BMPR1A, CDK8, MDM2, EXT1, ACSL3, STK36, HMGA2, RUNX1T1, TLR4, ERCC5, THOC5, IDH2 and HNRNPA2B1.

13. The composition of claim 11, wherein the agent is a probe or primer set.

14. The composition of claim 11, wherein the breast cancer is a triple negative breast cancer.

15. A kit comprising the composition of claim 11 as an active ingredient.

16. (canceled)

17. A method for predicting the responsiveness of a breast cancer patient to chemotherapy, the method comprising:
obtaining a sample of a test subject undergoing chemotherapy;
extracting genomic DNA from the sample;
confirming the presence of absence of the deletion of a gene in the extracted genomic DNA; and
determining that the test subject has a breast cancer with a poor prognosis in case the presence of the deletion of a gene is confirmed in the genomic DNA.

18. The method of claim 17, wherein the chemotherapy is an adjuvant chemotherapy.

19. The method of claim 17, wherein the gene is at least one gene selected from the group consisting of ATM, CHUK, EPHA5, LIFR, EBF1, NR4A3, MITF, TRIM33, MAP2K4, BMPR1A, CDK8, MDM2, EXT1, ACSL3, STK36, HMGA2, RUNX1T1, TLR4, ERCC5, THOC5, IDH2 and HNRNPA2B1.

20. The composition of claim 11, wherein the composition is used for predicting the prognosis of a breast cancer patient or predicting the responsiveness of a breast cancer patient to chemotherapy.

21. (canceled)


SEQUENCE LISTING (21 sequences)

SEQ ID NO: 1 - DNA, 20 bp, Artificial Sequence
  WRN_CNV_F
ccaggtctctgtgcatttca

SEQ ID NO: 2 - DNA, 25 bp, Artificial Sequence
  WRN_CNV_R
ggtaatacctgaaaacaggaactga

SEQ ID NO: 3 - DNA, 22 bp, Artificial Sequence
  WRN_Probe
gaaatgatgaaaaagcaacaca

SEQ ID NO: 4 - DNA, 27 bp, Artificial Sequence
  ATM_CNV#1_F
gaataattgtttttatttctttgttgc

SEQ ID NO: 5 - DNA, 21 bp, Artificial Sequence
  ATM_CNV#1_R
ttaacaatcgcaggaaaaagc

SEQ ID NO: 6 - DNA, 23 bp, Artificial Sequence
  ATM_CNV#1_probe
tgtcttaattgcagaagagtcca

SEQ ID NO: 7 - DNA, 23 bp, Artificial Sequence
  ATM_CNV#2_F
aaacaaaagtgttgtcttcatgc

SEQ ID NO: 8 - DNA, 23 bp, Artificial Sequence
  ATM_CNV#2_R
gaacttctttttcaccagtgtgg

SEQ ID NO: 9 - DNA, 20 bp, Artificial Sequence
  ATM_CNV#2_probe
tgcagttatccaagatggca

SEQ ID NO: 10 - DNA, 22 bp, Artificial Sequence
  BRCA1_CNV#1_F
ttctacagagtgaacccgaaaa

SEQ ID NO: 11 - DNA, 19 bp, Artificial Sequence
  BRCA1_CNV#1_R
ggctaaggcaggaggactg

SEQ ID NO: 12 - DNA, 20 bp, Artificial Sequence
  BRCA1_CNV#1_probe
atggagtcttgctctgtggc

SEQ ID NO: 13 - DNA, 20 bp, Artificial Sequence
  BRCA1_CNV#2_F
cagcgatactttcccagagc

SEQ ID NO: 14 - DNA, 20 bp, Artificial Sequence
  BRCA1_CNV#2_R
ttgcaaaaccctttctccac

SEQ ID NO: 15 - DNA, 20 bp, Artificial Sequence
  BRCA1_CNV#2_probe
tgctgaagaccccaaagatc

SEQ ID NO: 16 - DNA, 26 bp, Artificial Sequence
  BRCA2_CNV#1_F
tccaaagattcagaaaactactttga

SEQ ID NO: 17 - DNA, 20 bp, Artificial Sequence
  BRCA2_CNV#1_R
gaatgtgtggcatgacttgg

SEQ ID NO: 18 - DNA, 22 bp, Artificial Sequence
  BRCA2_CNV#1_probe
tggaagatgatgaactgacaga

SEQ ID NO: 19 - DNA, 20 bp, Artificial Sequence
  BRCA2_CNV#2_F
cattgatggacatggctctg

SEQ ID NO: 20 - DNA, 22 bp, Artificial Sequence
  BRCA2_CNV#2_R
tgaaaggcaaaaattcatcaca

SEQ ID NO: 21 - DNA, 22 bp, Artificial Sequence
  BRCA2_CNV#2_probe
caaaaacaactccaatcaagca